(19) United States
(12) Patent Application Publication
(10) Pub. No.: US 2024/0201175 A1
(43) Pub. Date: Jun. 20, 2024

(54) METHODS OF DETERMINING A TREATMENT PROTOCOL FOR AND/OR A PROGNOSIS OF A PATIENT’S RECOVERY FROM A BRAIN INJURY RESULTING FROM A HYPOXIC EVENT

(71) Applicant: Quanterix Corporation, Billerica, MA (US)

(72) Inventors: David Wilson, Boxborough, MA (US); Linan Song, Waltham, MA (US)

(73) Assignee: Quanterix Corporation, Billerica, MA (US)

(21) Appl. No.: 18/389,900

(22) Filed: Dec. 20, 2023

Related U.S. Application Data
(62) Division of application No. 14/111,331, filed on Dec. 18, 2013.
(60) Provisional application No. 61/474,315, filed on Apr. 12, 2011.

(51) Int. Cl. G01N 33/53; G01N 33/68

(52) U.S. Cl. CPC G01N 33/5306; G01N 33/6896; G01N 2333/4709; G01N 2800/28; G01N 2800/2871; G01N 2800/52

(57) ABSTRACT

The present invention, in some embodiments, generally relates to methods of determining a treatment protocol for and/or a prognosis of a patient's recovery from a brain injury resulting from a hypoxic event. In some embodiments, methods are provided for determining a measure of the concentration of beta-amyloid peptide in a patient sample containing or suspected of containing beta-amyloid peptide.

### RELATED APPLICATIONS

This application is a division of U.S. application Ser. No. 14/111,331, filed Dec. 18, 2013, and entitled “METHODS OF DETERMINING A TREATMENT PROTOCOL FOR AND/OR A PROGNOSIS OF A PATIENT'S RECOVERY FROM A BRAIN INJURY RESULTING FROM A HYPOXIC EVENT”, which is a national stage of International Patent Application Serial No. PCT/US2012/033338, filed Apr. 12, 2012, and entitled “METHODS OF DETERMINING A TREATMENT PROTOCOL FOR AND/OR A PROGNOSIS OF A PATIENT'S RECOVERY FROM A BRAIN INJURY RESULTING FROM A HYPOXIC EVENT,” which claims priority under 35 U.S.C. § 119(c) to U.S. Provisional Patent Application Ser. No. 61/474,315, filed Apr. 12, 2011, and entitled “METHODS OF DETERMINING A TREATMENT PROTOCOL FOR AND/OR A PROGNOSIS OF A PATIENT'S RECOVERY FROM A BRAIN INJURY RESULTING FROM A HYPOXIC EVENT,” which are incorporated herein by reference in their entireties for all purposes.

### FIELD OF THE INVENTION

The present invention generally relates, in some embodiments, to methods of determining a treatment protocol for and/or a prognosis of a patient's recovery from a brain injury resulting from a hypoxic event. In some embodiments, methods are provided for determining a measure of the concentration of beta-amyloid peptide in a patient sample containing or suspected of containing beta-amyloid peptide.

### BACKGROUND OF THE INVENTION

Hypoxia generally refers to a deficiency in the amount of oxygen reaching body tissues or a condition of insufficient levels of oxygen in tissue or blood. Oxygen deprivation to the brain results in neuronal damage and death, which is in turn related to the extent of long term brain dysfunction. The concentration of certain biomarkers may become elevated under hypoxic conditions. For example, certain such biomarkers have been found to become elevated in the brain and central nervous system under hypoxic condition. However, since such biomarkers must diffuse across the blood brain barrier, they may be present in the blood in proportion in extremely low concentrations that are not reliably measurable by typical conventional immunoassays. While the concentration of some biomarkers in the brain and central nervous system are known to increase with hypoxic events, the increased concentration has not been correlated with specific diagnostic indications and/or methods of treatment. In addition, while some methods exist for determining a cerebral hypoxic event in a patient and/or determining a course of treatment following a hypoxic event, many of the known methods are costly (e.g., magnetic resonance imaging) and/or provide unclear results and/or predictors. Accordingly, improved methods are needed.

### SUMMARY OF THE INVENTION

In some embodiments, a method for determining a measure of the concentration of beta-amyloid peptide in a patient sample containing or suspected of containing beta-amyloid peptide comprises performing an assay to determine a measure of the concentration of beta-amyloid peptide in the sample, wherein the limit of detection of beta-amyloid peptide of the assay is less than about 0.2 pg/mL.

In some embodiments, a method for determining a patient's prognosis for recovery from, and/or determining a course of treatment for, a brain injury following a hypoxic event or suspected hypoxic event comprises performing an assay on a plurality of samples obtained from the patient following the event to determine a measure of the concentration of at least one biomarker in each sample, wherein the concentration of the biomarker in each sample is less than about 100 pg/mL, and wherein the assay has a limit of quantification of less than about 10 pg/mL; and determining a prognosis of the patient's recovery and/or determining a course of treatment based at least in part on the measured concentration of the at least one biomarker measured in each sample.

In some embodiments, a method for determining a patient's prognosis for recovery from, and/or determining a course of treatment for, a brain injury following a hypoxic event or suspected hypoxic event comprises determining a prognosis of the patient's recovery and/or determining a course of treatment based at least in part on a measured concentration of at least one biomarker measured in each sample by an assay performed on a plurality of samples obtained from the patient following the event to determine the measure of the concentration of the at least one biomarker in each sample, wherein the concentration of the biomarker in each sample is less than about 100 pg/mL, and wherein the assay has a limit of quantification of less than about 10 pg/mL.

In some embodiments, a method for performing an assay and providing data for determining a patient's prognosis for recovery from, and/or determining a course of treatment for, a brain injury following a hypoxic event or suspected hypoxic event, comprises performing an assay on a plurality of samples obtained from the patient following the event to determine a measure of the concentration of at least one biomarker in each sample, wherein the concentration of the biomarker in each sample is less than about 100 pg/mL, and wherein the assay has a limit of quantification of less than about 10 pg/mL; and providing data from the assay to enable determining a prognosis of the patient's recovery and/or determining a course of treatment based at least in part on the measured concentration of the at least one biomarker measured in each sample.

In some embodiments, a method for determining a patient's prognosis for recovery from, and/or determining a course of treatment for, a brain injury following a hypoxic event or suspected hypoxic event comprises determining a measure of the concentration of a biomarker in each of a plurality of sample obtained from the patient following the event, wherein the assay has a limit of quantification of less than about 10 pg/mL, and wherein the limit of quantification of the assay used to determine the measure of concentration is less than about 10 pg/mL; and determining a prognosis of the patient's recovery and/or determining a course of treatment based at least in part on the measured concentration of the at least one biomarker measured in each sample. In some embodiments, a method of determining a treatment protocol for, and/or a prognosis of a patient's recovery from, a brain injury resulting from a hypoxic event comprises performing an assay to determine a measure of the concentration of only a first biomarker present in a plurality of samples obtained from the patient following the event, wherein the concentration of the first biomarker is less than about 100 pg/mL, and wherein the assay has a limit of quantification of less than about 10 pg/mL; and determining a prognosis of the patient's recovery from the brain injury and/or a method of treatment based at least in part on the measured concentration of the first biomarker in the plurality of samples.

In some embodiments, a method of determining a treatment protocol for, and/or a prognosis of a patient's recovery from, a brain injury resulting from a hypoxic event comprises determining a prognosis of the patient's recovery from the brain injury and/or a method of treatment based at least in part on a measured concentration of a first biomarker in the plurality of samples, wherein the measured concentration has been determined by performing an assay to measure the concentration of only the first biomarker present in a plurality of samples obtained from the patient following the event, wherein the concentration of the first biomarker is less than about 100 pg/mL, and wherein the assay has a limit of quantification of less than about 10 pg/mL.

In some embodiments, a method for performing an assay and providing data for determining a treatment protocol for, and/or a prognosis of a patient's recovery from, a brain injury resulting from a hypoxic event comprises performing an assay to determine a measure of the concentration of only a first biomarker present in a plurality of samples obtained from the patient following the event, wherein the concentration of the first biomarker is less than about 100 pg/mL, and wherein the assay has a limit of quantification of less than about 10 pg/mL; and providing data from the assay to enable determining a prognosis of the patient's recovery from the brain injury and/or a method of treatment based at least in part on the measured concentration of the first biomarker in the plurality of samples.

In some embodiments, a method of determining a treatment protocol for, and/or a prognosis of a patient's recovery from, a brain injury resulting from a hypoxic event comprises determining a measure of the concentration of only a first biomarker in each of a plurality of sample obtained from the patient following the event wherein the concentration of the first biomarker in the samples is less than about 100 pg/mL, and wherein the limit of quantification of the assay used to determine the measure of concentration is less than about 10 pg/mL; and determining a prognosis of the patient's recovery from the brain injury and/or a method of treatment based at least in part on the measured concentration of the first biomarker in the plurality of samples.

In some embodiments, a method of determining a treatment protocol for and/or a prognosis of a patient's recovery from a brain injury resulting from a hypoxic event comprises performing an assay on a blood sample from the patient and/or plasma and/or serum derived from the blood sample to determine a measure of the concentration of beta-amyloid peptide in the sample, wherein the concentration of the biomarker is less than about 500 pg/mL, and wherein the assay has a limit of quantification of less than about 10 pg/mL; and determining a prognosis of the patient's recovery from the brain injury and/or a method of treatment based at least in part on the measured concentration of beta-amyloid peptide present in the sample.

In some embodiments, a method of determining a treatment protocol for and/or a prognosis of a patient's recovery from a brain injury resulting from a hypoxic event comprises determining a prognosis of the patient's recovery from the brain injury and/or a method of treatment based at least in part on a measured concentration of beta-amyloid peptide present in a patient sample, wherein the measured concentration has been determined by performing an assay on the patient sample, which comprises a blood sample from the patient and/or plasma and/or serum derived from the blood sample, to determine the measure of the concentration of beta-amyloid peptide in the sample, wherein the concentration of the biomarker is less than about 500 pg/mL, and wherein the assay has a limit of quantification of less than about 10 pg/mL.

In some embodiments, a method for performing an assay and providing data for determining a treatment protocol for and/or a prognosis of a patient's recovery from a brain injury resulting from a hypoxic event comprises performing an assay on a blood sample from the patient and/or plasma and/or serum derived from the blood sample to determine a measure of the concentration of beta-amyloid peptide in the sample, wherein the concentration of the biomarker is less than about 500 pg/mL, and wherein the assay has a limit of quantification of less than about 10 pg/mL; and providing data from the assay to enable determining a prognosis of the patient's recovery from the brain injury and/or a method of treatment based at least in part on the measured concentration of beta-amyloid peptide present in the sample.

In some embodiments, a method of determining a treatment protocol for and/or a prognosis of a patient's recovery from a brain injury resulting from a hypoxic event comprises determining a measure of the concentration of beta-amyloid in each of a plurality of samples obtained from the patient following the event wherein the concentration of the first biomarker in the samples is less than about 500 pg/mL, and wherein the limit of quantification of the assay used to determine the measure of concentration is less than about 10 pg/mL; and determining a prognostic of the patient's recovery from the brain injury and/or a method of treatment based at least in part on the measured concentration of beta-amyloid peptide present in the sample.

In some embodiments, a method of determining a method of treatment for and/or a prognosis of a patient's recovery from a brain injury resulting from a hypoxic event comprises (a) performing an assay on each of a plurality of samples obtained from the patient following the event to determine the measured concentration of beta-amyloid peptide in each of the samples, wherein the plurality of samples are obtained from the patient over a period of time of at least about 48 hours; (b) determining a baseline concentration of beta-amyloid peptide for the patient, the maximum concentration of beta-amyloid peptide concentration in a single sample obtained during the period of time, and the duration of time in which the concentration of beta-amyloid peptide is increasing in the samples obtained from the patient during the period of time; and (c) determining a prognosis of the patient's recovery from the brain injury and/or a method of treatment based at least in part on the baseline concentration, the maximum concentration, and/or the duration of time determined in step (b).

In some embodiments, a method of determining a method of treatment for and/or a prognosis of a patient's recovery from a brain injury resulting from a hypoxic event comprises determining a prognosis of the patient's recovery from the brain injury and/or a method of treatment based at least in part on the baseline concentration, the maximum concentration, and/or the duration of time determined, which has been determined by: (a) performing an assay on each of a plurality of samples obtained from the patient following the event to determine the measured concentration of beta-amyloid peptide in each of the samples, wherein the plurality of samples have been obtained from the patient over a period of time of at least about 48 hours and (b) determining the baseline concentration of beta-amyloid peptide for the patient, the maximum concentration of beta-amyloid peptide concentration in a single sample obtained during the period of time, and the duration of time in which the concentration of beta-amyloid peptide is increasing in the samples obtained from the patient during the period of time.

In some embodiments, a method for performing an assay and providing data for determining a method of treatment for and/or a prognosis of a patient's recovery from a brain injury resulting from a hypoxic event comprises (a) performing an assay on each of a plurality of samples obtained from the patient following the event to determine the measured concentration of beta-amyloid peptide in each of the samples, wherein the plurality of samples are obtained from the patient over a period of time of at least about 48 hours; (b) determining a baseline concentration of beta-amyloid peptide for the patient, the maximum concentration of beta-amyloid peptide concentration in a single sample obtained during the period of time, and the duration of time in which the concentration of beta-amyloid peptide is increasing in the samples obtained from the patient during the period of time; and (c) providing data derived in steps (a) and (b) to enable determining a prognosis of the patient's recovery from the brain injury and/or a method of treatment based at least in part on the baseline concentration, the maximum concentration, and/or the duration of time.

### BRIEF DESCRIPTION OF THE DRAWINGS

FIG. 1a is a schematic flow diagram depicting one embodiment of steps (A-D) for performing an exemplary method of the present invention;

FIG. 1b is a schematic flow diagram depicting one embodiment of steps (A-D) for performing an exemplary method of the present invention;

FIG. 2 shows plots of a measure of the concentration of beta-amyloid (1-42) versus time for patients having (a-c) a good outcome and (d-f) a bad outcome;

FIG. 3 shows a plot of a measure of the concentration of beta-amyloid (1-42) versus time including various parameters that may be correlated with a prognostic indication and/or a method of treatment;

FIG. 4 shows an exemplary calibration dose-response of an assay method for determining a measure of the concentration of beta-amyloid (1-42) up to 250 pg/mL;

FIG. 5 provides a plot showing day-to-day reproducibility for determining a measure of the concentration of beta-amyloid (1-42), according to some embodiments of the present invention;

FIG. 6 shows plots of a measure of the concentration of beta-amyloid (1-42) in plasma and CSF samples from Alzheimer's, MCI, and normal control subjects; and

FIG. 7 shows a plot of a measure of the concentration of beta-amyloid (1-42) versus time for an exemplary patient.

Other aspects, embodiments, and features of the invention will become apparent from the following detailed description when considered in conjunction with the accompanying drawings. The accompanying figures are schematic and are not intended to be drawn to scale. For purposes of clarity, not every component is labeled in every figure, nor is every component of each embodiment of the invention shown where illustration is not necessary to allow those of ordinary skill in the art to understand the invention. All patent applications and patents incorporated herein by reference are incorporated by reference in their entirety. In case of conflict, the present specification, including definitions, will control.

### DETAILED DESCRIPTION

The present invention generally relates to methods of determining a treatment protocol for and/or a prognosis of a patient's recovery from a brain injury resulting from a hypoxic event. In some embodiments, methods are provided for determining a measure of the concentration of beta-amyloid peptide in a patient sample containing or suspected of containing beta-amyloid peptide. In some cases, the beta-amyloid peptide is beta-amyloid (1-42). The subject matter of the present invention involves, in some cases, interrelated products, alternative solutions to a particular problem, and/or a plurality of different uses of one or more systems and/or articles.

In some embodiments, a method of the present invention comprises determining a measure of the concentration of at least one biomarker in one or more samples obtained from a patient following a hypoxic event (or a suspected hypoxic event). A prognostic indication of the patient's recovery and/or determining a course of treatment may be based at least in part on the measure of the concentration of the at least one biomarker present in the one or more samples. It should be understood, that while much of the discussion below is directed to methods involving the analysis of more than one sample, this is by way of example only, and similar methods may be employed wherein only a single sample is employed.

As will be known to those of ordinary skill in the art, the term “hypoxia” generally refers to a deficiency in the amount of oxygen reaching body tissues or a condition of insufficient levels of oxygen in tissue or blood. Hypoxia at a cellular level develops when delivery of oxygen to cell mitochondria slows as the partial pressure gradient from capillaries to tissues decreases. As the delivery of oxygen decreases, aerobic metabolism stops and less efficient anaerobic pathways of glycolysis become responsible for the production of cellular energy. The end result is an increase in cellular concentrations of sodium, calcium, and hydrogen ions which may lead to cell death.

Oxygen deprivation to the brain results in neuronal damage and death. The extent of neuronal damage and death in turn relates to the extent of long term brain dysfunction, as can be assessed using standard criteria (such as Cerebral Performance Category, CPC rating, or like criteria). Severe hypoxia can result in a patient's death and/or an irreversible brain injury (e.g., resulting in the patient being in a vegetative state). Hypoxic events may be global (e.g., due to low oxygen content in the blood) or focused (e.g., affecting only an area of the brain). Causes of hypoxia include, but are not limited to, local asphyxia (e.g., caused by smoke inhalation), carbon monoxide poisoning and/or toxicity, cardiac arrest, choking, drowning, high altitudes, strangulation, an ischemic event, thrombosis, arterial embolism, hemorrhage, swelling of the brain, stroke, physical trauma and/or physical injury (e.g., blunt trauma to the head), arteriosclerosis, and/or atherosclerosis. In some cases, the event may be myocardial infarction, myocardial ischemia, and/or transient ischemic attack.

Hypoxic conditions can lead to the production and/or change in the concentration of certain biomarkers. That is, the concentration of certain biomarkers increase or decrease following the hypoxic event. For example, the production of the proteolytic products of β-amyloid precursor protein has been found to become elevated in the brain and central nervous system under hypoxic condition. The increased concentration is theorized to be due to a hypoxia-inducible factor (HIF-1) that promotes the production of beta-amyloid peptides from amyloid precursor protein, a membrane protein concentrated in neuronal synapses. A cascade of beta-amyloid peptides, including beta-amyloid (1-42), is generated in the brain in proportion to the extent of hypoxia. Beta-amyloid peptides could in turn diffuse across the blood brain barrier and into the blood in proportion to the extent of hypoxia, and may be generally found in low abundance. The ability to determine a change in the concentration of a biomarker in a plurality of samples (or a single sample) obtained from a patient following a hypoxic event can, in some embodiments, be correlated with a prognostic indication of the patient's recovery from a brain injury and/or used to determine a method of treatment. In some cases, sample(s) of the patient's cerebrospinal fluid (CSF) may be obtained and analyzed to determine the concentration and/or a change in the concentration of the biomarker. In some cases, however, it is advantageous to determine the level of a biomarker in the blood of a patient as compared to CSF, as blood sampling is generally less invasive and may result in fewer complications as compared to CSF sampling. However, many of the biomarkers that are present in the CSF have a slow rate of transmission across and/or a high barrier of transportation across the blood-brain barrier (BBB) and thus, the concentration of the biomarker in the patient's blood may be sufficient lowered as compared to the concentration in CSF to make it difficult or impossible to accurately determine using typically employed conventional immunoassays. Accordingly, assay methods which have very low limits of quantification (LOQ) and/or limits of detection (LOD) are generally necessary to determine a measure of the concentration of a biomarker in the patient's blood to provide statistically significant and/or meaningful results. In some embodiments, the methods of the present invention make use of methods having very low LODs and/or LOQs (e.g., in the low pg/mL range) to determine a measure of the concentration of a biomarker in a sample(s) obtained from a patient following a hypoxic event. Various parameters related to the changes in the concentration of the biomarker in the samples (e.g., blood samples) may be correlated with a prognostic indication and/or a method of treatment following a hypoxic event. Correlations (e.g., between the concentration and prognostic indication(s) and/or between the concentration and method(s) of treatment) have been discovered and/or are now discoverable due to recent advancements in technology which allow for the determination of the low concentrations of biomarkers in bodily fluids with sufficient accuracy and precision, thus allowing for the variations in concentration to be statistically significant and therefore, diagnostic.

In embodiments where a plurality of samples are obtained from a patient, the samples may be obtained from a patient over any suitable period of time. Generally, the period of time may be selected such that the concentration of a biomarker in the samples becomes statistically significant and/or a trend is observable (e.g., an increase and/or decrease in the concentration). For biomarkers which are analyzed in blood samples, the period of time over which a plurality of samples are obtained from the patient may account for any lag time required for the biomarker to cross the BBB. Non-limiting examples of suitable periods of time in which the samples may be obtained from the patient include 1 hour, 2 hours, 3 hours, 4 hours, 6 hours, 8 hours, 10 hours, 12 hours, 18 hours, 24 hours, 36 hours, 48 hours, 60 hours, 72 hours, 4 days, 5 days, 6 days, 7 days, or more. In some cases, the duration of time of sample collection time is at least 60 hours, or at least 72 hours. In some cases, the duration of time of sample collection is between 12 hours and 7 days, or between 24 hours and 4 days, or between 2 days and 4 days, or between 3 days and 4 days. The first sample may be obtained from the patient without a short timeframe following the hypoxic event. For example, the first sample may be obtained from the patient within 1 hour, 2 hours, 3 hours, 4 hours, 5 hours, 6 hours, 8 hours, 10 hours, or 12 hours of the hypoxic event. In some cases, the first sample is obtained within 6 hours of the hypoxic event. In some embodiments, a first sample is obtained from the patient within 6 hours of the suspected hypoxic event, and at about 1, about 2, about 6, about 12, about 24, about 48, and about 72 hours, following the first sampling. In some embodiments, additional samples are obtained at about 96 and/or at about 108 hours following the first sampling.

Any number of samples (e.g., one or more) may be obtained from the patient over the time period of sample collection. Generally, the minimum number of samples obtained is such that a trend (e.g., an increase or decrease) in the concentration of the biomarker is observable. Non-limiting examples of the number of samples that are obtained from the patient (e.g., during the prescribed collection time) is at least about bout 1, about 2, at least about 3, at least about 4, at least about 5, at least about 6, at least about 7, at least about 8, at least about 9, at least about 10, at least about 12, at least about 15 or more. In some cases, the number of samples obtained from the patient is between 2 and 20, between 5 and 15, or between 5 and 10.

The sample(s) obtained from the patient may be from any suitable bodily source. In some cases, the samples are CSF samples or fluid samples. In some cases, the samples are not CSF fluid samples. In some cases, the samples are blood or blood products (e.g., whole blood, plasma, serum, etc.). In other cases, the samples may be urine or saliva samples. In some embodiments, the samples may be analyzed directly (e.g., without the need for extraction of the biomarker from the fluid sample) and/or with dilution (e.g., addition of a buffer or agent to the sample). Generally, each of the samples obtained from the patient is collected using substantially similar procedures (e.g., to ensure minimal variation between samples based on sample collection methods). Those of ordinary skill in the art will be aware of suitable systems and methods for obtaining a sample from a patient.

Each of or substantially all of the samples may be analyzed using an assay method (e.g., as described herein) to determine a measure of the concentration of at least one biomarker in each, a subset of or substantially all of the samples. In some cases, the methods comprise determining a measure of the concentration of a single biomarker in the samples. In other cases, a method comprises determining a measure of the concentration of more than one biomarker in each, a subset of or substantially all of the samples. For example, a measure of the concentration of 2, 3, 4, 5, 6, 7, 8, 9, 10, or more, biomarkers may be determined in the samples.

In some embodiments, the methods of the present invention comprise determining a prognostic indication based at least in part on the measured concentration of the at least one biomarker in the samples. In some cases, the prognostic indication may be correlated with standard criteria employed to define long-term brain dysfunction and/or injury. Those of ordinary skill in the art will be aware of such criteria, for example, cerebral performance category ratings (“CPC rating”), or more specifically, Glasgow-Pittsburgh cerebral performance category ratings (or scale) (e.g., see Teasdale G, Jennett B (1974); Assessment of coma and impaired consciousness; Lancet 2 (7872): 81-84). The CPC scale ranges from 1 to 5, with 1 representing a slight possibility of neurological deficit and 5 representing severe deficit and/or death. In some methods of the present invention, the prognostic indication of the patient's recovery from the brain injury is classified as either “good” (e.g., correlating to a CPC score of 1 or 2) corresponding to a high likelihood of recovery and/or returning to independent living, or “poor” (e.g., correlating to a CPC score of 3, 4, or 5) corresponding to little possibility of a full recovery and resulting in assisted living and/or death.

In the CPC scale, a rating of 1 is generally classified as good cerebral performance. The patient is conscious and alert, is able to work, but may have mild neurological or psychological deficit. A rating of 2 is generally classified as having moderate cerebral disability. The patient is conscious and has sufficient cerebral function for independent activities of daily life, and is generally able to work in sheltered environment. A rating of 3 corresponds to severe cerebral disability. While the patient is conscious, they generally depend on others for daily support because of impaired brain function. The patient may have abilities ranging from ambulatory state to severe dementia or paralysis. A rating of 4 corresponds to a coma or vegetative state. The patient is generally unaware, even if they appear awake (e.g., the patient is in a vegetative state) without interaction with environment and is cerebral unresponsive. A rating of 5 refers to brain death, associated with apnea, areflexia, and/or EEG silence. The CPC scale is summarized in Table 1.

TABLE 1
CPC Scale Summary

| CPC Score | Description |
|---|---|
| 1 | Conscious and alert with normal function or |
| | only slight disability |
| 2 | Conscious and alert with moderate disability |
| 3 | Conscious with severe disability |
| 4 | Comatose or persistent vegetative state |
| 5 | Brain dead or death from other causes |

Other non-limiting examples of suitable criteria include the “scale g” criteria (e.g., see Dekaban AS, Robinson CE.; Application of a new rating scale of brain dysfunction to monitoring rehabilitation in 65 patients with severe head injury, Bull Clin Neurosci., 1984; 49, 82-92), the “Rancho Los Amigos Scale,” and the “Disabilities Rating Scale.”

In addition to the biomarkers specifically mentioned herein, those of ordinary skill will be aware of other suitable biomarkers to use in connection with the methods described herein. As described herein, the biomarker generally undergoes a change in concentration as a result of a hypoxic event. For example, the concentration of the biomarker may increase or decrease as a result of the hypoxic event. Non-limiting examples of biomarkers include neuron specific neuronal enolase (NSE), β-site aPP-cleaving enzyme 1 (BACe1), S100B, myelin basic protein (MBP), growth associated protein 43, glutamine synthetase, glial fibrillary acid protein (GFAP), glycine transporter (e.g., GLYT1, GLYT2), neuron specific glycoprotein (e.g., GP50), calpain, neurofibrillary protein, heat shock protein 72, beta-amyloid precursor proteins, calbindin D-28K, proteolipid protein, myeline associated glycoprotein, neurofilament H, creatine kinase protein (e.g., CK-BB), tau proteins (including phosphorylated taus such as p-tau-81 or p-tau-231), and endothelium membrane proteins (e.g., thrombomodulin).

In some cases, the biomarker may be a beta-amyloid peptide. Beta-amyloid peptides are known to those of ordinary skill in the art (e.g., see Blennow et al., Nature Reviews, Neurology, 6, 2010, 131; Portelius et al., Neurobiology of Aging, 2009, Corrected Proof in Press, doi: 10.1016/j.neurobiolaging.2009.06.002). Beta-amyloid peptides are formed by sequential cleavage of the amyloid precursor protein (APP). APP can be processed by alpha-, beta-, and gamma-secretases, wherein the beta-amyloid peptide is generated by successive actions of the beta and gamma secretases. Various forms of beta-amyloid peptides may be contemplated for use as a target with the methods described herein, include isoforms and short isoforms (e.g., ranging from beta-amyloid (1-14) up to beta-amyloid (1-43)). Beta-amyloid isoforms can have varying lengths of amino acids, with beta-amyloid (1-40) and beta-amyloid (1-42) being the most common. Non-limiting examples of isoforms of beta-amyloid peptides include beta-amyloid (1-40), beta-amyloid (1-42), beta-amyloid (1-37), beta-amyloid (1-38) and beta-amyloid (1-39). Non-limiting examples of short beta-amyloid isoforms include beta-amyloid (1-14), beta-amyloid (1-15) and beta-amyloid (1-16). In some embodiments, the biomarker is beta-amyloid (1-42).

As will be know, mild ischemia due to arteriolosclerosis is common in the elderly, and more common in Alzheimer's patients, an arteriolosclerosis could result in chronic upregulation of beta amyloid (1-42), which could lead to plaque deposits in the brain. In certain embodiments, the methods of the invention are performed on subjects not diagnosed with or suspected of suffering from Alzheimer's disease and the prognosis determined/provided by the methods disclosed herein does not relate to Alzheimer's disease. In other embodiments, the methods of the invention and the prognosis determined/provided by the methods disclosed herein may be used in the context of Alzheimer's disease.

The plurality of samples obtained from the patient may be analyzed (e.g., using an assay method as described herein) to determine a measure of the concentration of the at least one biomarker in each of the samples (or a single sample). A prognostic indication and/or a method of treatment may be determined based at least in part on the measure of the concentration of the biomarker in each of the plurality of samples. The data may be analyzed using a variety of techniques, as described herein, and a prognostic indication for recovery from a brain injury (e.g., a “good” outcome or a “poor” outcome), or a specific method of treatment may be determined based on the results.

In some embodiments, the measure of the concentration of the at least one biomarker for each of the plurality of samples obtained from the patient may be plotted on a graph of concentration versus time (e.g., in hours), and one or more parameters can be obtained from the graph and used to determine the method of treatment and/or the prognostic indication. Non-limiting examples of parameters that may be determined and/or employed include baseline biomarker concentration, increase in biomarker concentration, duration of rise of biomarker concentration, maximum slope of increasing biomarker concentration, rate of change of biomarker concentration, area under the curve and/or magnitude of the fold increase of biomarker concentration. Each of the parameters and their determination will now be described in detail, followed by a description of possible data analysis and methods useful or potentially useful to determine suitable correlations between the measure of the concentration determined in the samples and a treatment/prognostic indication. The measure of the concentration of the biomarker may be determined/displayed in any suitable unit. In some cases, the measure of the concentration is determined/displayed in pg/mL.

The term “baseline biomarker concentration” refers to the concentration of the biomarker generally present in a fluid sample from a normal patient (e.g., prior to or unexposed to a hypoxic event). The baseline biomarker concentration can be determined using a variety of methods which will be commonly known and understood by those of ordinary skill in the art. In some cases, the baseline biomarker concentration can be determined by averaging the value of a biomarker present in a population of control patients (e.g., patients who have not experienced a hypoxic event). This may be useful in embodiments where the baseline concentration of the biomarker is substantially the same for a given population of individuals (e.g., based on age, gender, medical condition, etc). However, many of the methods described herein require accurate determination of extremely low concentrations of the at least one biomarker in samples obtained from a patient, and accordingly, a general baseline concentration of a biomarker (e.g., based on a sampling of a population) may not provide enough accuracy and/or precision to give useful results. Accordingly, a measure of the baseline biomarker concentration, in some embodiments, may be determined for each individual patient. In some embodiments, the baseline biomarker concentration may be set to zero, e.g., in embodiments where the concentration of the biomarker is zero or essentially zero in normal patients.

In some cases, the baseline biomarker concentration for each individual is equal to the measure of the concentration of the biomarker in the first sample obtained from the patient following the hypoxic event. In other cases, if available, a baseline biomarker concentration is the measure of the concentration of the biomarker present in a sample obtained from the patient prior to the hypoxic event. In yet other cases, the baseline biomarker concentration is the average concentration of the biomarker determined in a plurality of samples taken from the patient immediately or substantially immediately following the hypoxic event (e.g., prior to the concentration of the biomarker increasing due to the hypoxic event). That is, the concentration of the biomarker in the plurality of samples obtained from the patient in a selected time period following the hypoxic event may be averaged to determine the baseline biomarker concentration for that patient. As will be understood by those of ordinary skill in the art, in such cases, any sample which has a concentration level that significantly differs from the other concentration levels during that period of time (e.g., a data point/outlier having a concentration which deviates significantly from the other concentrations measured during the time frame) may be excluded from the averaging calculation. In some cases, if a sample has a concentration which differs by greater than about 50% from the calculated average, that sample may be excluded from the averaging calculation. In some cases, if a sample has a concentration which differs by more than about 0.5 pg/mL, about 1 pg/mL, about 2 pg/mL, about 5 pg/mL, or about 10 pg/mL from the calculated average, that sample may be excluded from the averaging calculation. An anomalous data point may be observed and/or caused by administration of drugs to the patient. The baseline biomarker concentration may be determined by averaging the concentration of the biomarker in the samples obtained from the patient between the first sample (e.g., obtained within 6 hours of the hypoxic event) and 24 hours following the first collection of a sample, or between the first sample and 18 hours following the first collection of a sample, or between the first sample, and 12 hours following the first collection of a sample, or between the first sample, and 6 hours following the first collection of a sample (e.g., not including any outliers).

The term “change in biomarker concentration” refers to the change (e.g., increase or decrease) in concentration of the biomarker over the time period in which the samples are collected. An increase in biomarker concentration may be calculated by subtracting the baseline biomarker concentration (e.g., as described herein) from the maximum biomarker concentration. The “maximum biomarker concentration” is the maximum measured concentration of the biomarker measured in a single sample collected over the duration of the sample collection. For example, if the duration of the sample collection is 72 hours, the maximum concentration is equal to the maximum measured concentration in a single sample which was obtained from the patient in the 72 hour period. A decrease in biomarker concentration may be calculated by subtracting an elevated biomarker concentration (e.g., as described herein) from the minimum biomarker concentration.

The term “magnitude of the fold increase of biomarker concentration” refers to the magnitude of the fold-increase in concentration over the duration of sample collection, and may be calculated by dividing the maximum biomarker concentration (e.g., as described herein) by the baseline biomarker concentration (e.g., as described herein).

The term “duration of rise of biomarker concentration” refers to the time over which the concentration of the biomarker is increasing during the time period over which the samples are collected. In some cases, the duration of rise of biomarker concentration can be determined by subtracting the time at which the last sample was collected at approximately the baseline biomarker concentration (e.g., starting time of the rise) from the time at which the maximum biomarker concentration was observed (e.g., ending time of the rise). That is, the duration of the rise may be equal to the ending time of the rise minus the starting time of the rise. The duration of rise is generally provided in hours.

The term “maximum slope of increasing biomarker concentration” refers to the slope at which the maximum increase in concentration occurred over the time period in which the samples were collected. Those of ordinary skill will be aware of methods to calculate this value using common graphical analysis methods. For example, a plot may be prepared showing concentration versus time, and the maximum slope may be calculated based on this plot. In some cases, the maximum slope is equal to the slope between two data points, whereas in other cases, the maximum slope may be determined based on the average slope between a plurality of data points.

The term “area under the curve,” (or AUC) is a common parameter used to analyze data, and such calculations will be well known and understood by those of ordinary skill in the art. In context with the methods of this disclosure, the AUC refers to the area under the biomarker concentration versus time curve. Generally the calculation takes into account the baseline biomarker concentration. Those of ordinary skill it the art will be aware of suitable algorithms and/or computer programs capable of determining the area under the curve for a selected set of data. In some cases, the area under the curve may be determined for more than one portion of the data. For example, a first area under the curve value may be determined for a first range of data, and a second area under the curve value may be determined for a second range of data. The first and the second ranges of data may or may not be overlapping (e.g., may comprise some overlapping data points or may comprise different data points). It should be understood, that determining the area under the curve can be accomplished using a variety of techniques which will be known to those of ordinary skill in the art, including but not limited to, plotting the concentration versus time on a graph and determining the area under the line/curve (e.g., optionally with use of a computer program) and/or determining a functional relationship between the concentration and time and integrating the data without requiring physically plotting or drawing of a graph. As used herein, the phrase “determining the area under the curve of a graph of a biomarker concentration versus time” covers all of the above techniques and others applicable for determining the area or equivalent, and is not limited to physically or electronically plotting the concentration versus time on a graph and determining the area under the line/curve.

The above parameters, alone or in combination, may be correlated to a prognostic indication and/or a method of treatment for a patient following a hypoxic event. Following determination of a correlation between biomarker concentrations and a prognostic indication and/or a method of treatment (e.g., using a plurality of samples obtained from a plurality of test patients with known outcomes), the correlation can be used in connection with methods to determine prognosis (e.g., prognostic indications) and/or methods of treatment for patients with unknown outcomes. That is, an algorithm can be developed relating changes in a biomarker concentration and specific methods of treatment and/or prognostic indications using samples from test patients having been subject to known methods of treatment and/or having a known prognosis. Once the algorithm has been developed, it can be used to determine methods of treatment and/or prognostic indications for patients with unknown outcomes.

To determine a correlation between a biomarker and a prognostic indication and/or a method of treatment (e.g., to develop an algorithm relating the measured biomarker concentration to preferred methods of treatment and/or prognostic indications), a plurality of samples from a plurality of test patients may be obtained. A “test patient” is a patient who has a known outcome (e.g., a good or a poor CPC score) and/or has received a certain treatment. The plurality of the samples obtained from each of the test patients may be analyzed to determine the measure of the concentration of at least one biomarker in the each of the samples. Some or all of the parameters described herein may be determined for each patient, and the data may be analyzed to determine correlations between the parameters and the patient outcomes and/or treatments, which can in turn be used to develop an algorithm. The algorithm may then be applied to the concentration of the at least one biomarker in samples obtained from a patient having an unknown outcome to determine a method of treatment and/or a prognostic indication for that patient. A specific example of such an analysis is described below, and further details are provided in Example 1.

Similar analysis and methods may be used to correlate suitable methods of treatment based upon the measured concentration(s) of a biomarker in a plurality of samples obtained from a patient. In some cases, the method of treatment may comprise administering at least one therapeutic agent to the patient. For example, the therapeutic agent may be a neuroprotective drug. Other non-limiting methods of treatment include administration of anti-oxidants, hypothermia, blood thinning, and administration of steroids (e.g., to help reduce brain swelling) (e.g., see T. S. Richmond, Cerebral Resuscitation After Global Brain Ischemia: Linking Research to Practice, American Association of Critical-Care Nurses Journal, May 1997, Volume 8, Number 2).

A therapeutic agent is generally administered in an amount effective to provide a medically desirable result. The effective amount will vary with the particular condition being treated, the age and physical condition of the subject being treated, the severity of the condition, the duration of the treatment, the nature of the concurrent therapy (if any), the specific route of administration and the like factors within the knowledge and expertise of the health care practitioner. In some cases, a therapeutic agent may reduce brain injuries resulting from the hypoxic event. In some cases, the method of treatment may involve a change in treatment, such as an increase or decrease in the dose of a therapeutic agent, a switch from one therapeutic agent to another therapeutic agent, an addition of another therapeutic agent to the existing therapeutic agent, or a combination thereof. A switch from one therapeutic agent to another may involve a switch to a therapeutic agent with a high risk profile but where the likelihood of expected benefit is increased.

In one embodiment, a method of the present invention for determining a treatment protocol for and/or a prognostic indication of a patient's recovery from a brain injury resulting from a hypoxic event comprises performing an assay on a plurality of samples to determine a measure of the concentration of beta-amyloid peptide in each sample and determining a prognostic indication of the patient's recovery from the brain injury and/or a method of treatment based at least in part on the measure of the concentration of beta-amyloid peptide present in the samples. In some embodiments, the beta-amyloid peptide is beta-amyloid (1-42). The sample, in some embodiments, is a blood sample from the patient and/or plasma and/or serum derived from the blood sample. In some cases, the concentration of the beta-amyloid in the samples is less than about or about 500 pg/mL, less than about or about 400 pg/mL, less than about or about 300 pg/mL, less than about or about 200 pg/mL, less than about or about 100 pg/mL, less than about or about 50 pg/mL, less than about or about 30 pg/mL, less than about or about 20 pg/mL, less than about or about 10 pg/mL, less than about or about 5 pg/mL, less than about or about 1 pg/mL, or less. In some cases, the assay has a limit of quantification of less than about or about 100 pg/mL, less than about or about 50 pg/mL, less than about or about 40 pg/mL, less than about or about 30 pg/mL, less than about or about 20 pg/mL, less than about or about 10 pg/mL, less than about or about 5 pg/mL, less than about or about 4 pg/mL, less than about or about 3 pg/mL, less than about or about 2 pg/mL, less than about or about 1 pg/mL, less than about or about 0.8 pg/mL, less than about or about 0.7 pg/mL, less than about or about 0.6 pg/mL, less than about or about 0.5 pg/mL, less than about or about 0.4 pg/mL, less than about or about 0.3 pg/mL, less than about or about 0.2 pg/mL, less than about or about 0.1 pg/mL, less than about or about 0.05 pg/mL, less than about or about 0.04 pg/mL, less than about or about 0.02 pg/mL, less than about or about 0.01 pg/mL, or less. In some cases, the assay has a limit of detection of less than about or about 100 pg/mL, less than about or about 50 pg/mL, less than about or about 40 pg/mL, less than about or about 30 pg/mL, less than about or about 20 pg/mL, less than about or about 10 pg/mL, less than about or about 5 pg/mL, less than about or about 4 pg/mL, less than about or about 3 pg/mL, less than about or about 2 pg/mL, less than about or about 1 pg/mL, less than about or about 0.8 pg/mL, less than about or about 0.7 pg/mL, less than about or about 0.6 pg/mL, less than about or about 0.5 pg/mL, less than about or about 0.4 pg/mL, less than about or about 0.3 pg/mL, less than about or about 0.2 pg/mL, less than about or about 0.1 pg/mL, less than about or about 0.05 pg/mL, less than about or about 0.04 pg/mL, less than about or about 0.02 pg/mL, less than about or about 0.01 pg/mL, or less.

In one embodiment, a correlation was determined between certain parameters (e.g., maximum beta-amyloid peptide concentration, duration of increase of beta-amyloid concentration, and the magnitude of the fold increase of beta-amyloid concentration) and a good (e.g., CPC rating 1 or 2) and a poor (e.g., CPC rating of 3, 4, or 5) prognostic indication. To determine the correlation, an assay was carried out on each of the samples obtained from a plurality of test patients having undergone a hypoxic event (e.g., patients having a known outcome and/or having undergone a certain method of treatment following a hypoxic event). The first sample was taken within 6 hours of the hypoxic event, and additional samples were generally obtained at about 1, 2, 6, 12, 24, 48, and 72 hours following the first sample (and optionally, at 96 and/or 108 hours). Each of the test patients had a known prognostic outcome according to the CPC scale. For each test patient, a plot of the measured concentration of beta-amyloid in pg/mL versus time in hours was prepared. The data was analyzed and the sum of the increase in beta-amyloid concentration (in pg/mL), the duration of the increase of beta-amyloid concentration (in hours), and the magnitude of the fold increase of beta-amyloid concentration was determined to correlate with a “good” or “poor” prognosis for the patients. In particular, a sum of greater than 50 correlated with a poor prognosis (e.g., a CPC score of 3, 4, or 5) and a score of less than about 50 correlated with a good prognosis (e.g., a CPC score of 1 or 2). Accordingly, a correlation/algorithm was established between the varying parameters relating to concentration and a prognostic indication.

The developed correlation/algorithm can be applied to patients with unknown outcomes. That is, samples may be obtained from a patient and the concentration of beta-amyloid in each of the samples can be determined. The data may be analyzed to determine the increase in beta-amyloid concentration (in pg/mL), the duration of the increase of beta-amyloid concentration (in hours), and the magnitude of the fold increase of beta-amyloid concentration. If the sum of the three parameters is greater than 50, the prognostic indication for that patient is “poor,” and if the sum is less than 50, the prognostic indication is “good.” Using similar techniques, a variety of correlations may be determined for this beta-amyloid (e.g., analysis of the area under the curve) and/or other biomarkers.

### EXEMPLARY ASSAY METHODS AND SYSTEMS

Those of ordinary skill in the art will be aware of a variety of assay methods and systems that may be used in connection with the methods of the present invention. Generally, the methods employed have low limits of detection and/or limits of quantification as compared to bulk analysis techniques (e.g., ELISA methods). The use of assay methods that have low limits of detection and/or limits of quantification allows for correlations to be made between the various parameters discussed above and a method of treatment and/or diagnostic indication that may otherwise not be determinable and/or apparent. For example, in the method described above which correlates the sum of three parameters relating to beta-amyloid concentration to a prognostic indication of brain injury, the limits of detection, and/or limits of quantification needs to be substantially lower than the LOD and/or LOQ provided by common ELISA techniques.

The terms “limit of detection” (or LOD) and “limit of quantification” (or LOQ) are given their ordinary meaning in the art. The LOD refers to the lowest analyte concentration likely to be reliably distinguished from background noise and at which detection is feasible. The LOD as used herein is defined as three standard deviations (SD) above background noise. The LOQ refers to the lowest concentration at which the analyte can not only be reliably detected but at which some predefined goals for bias and imprecision are met. Generally, as is used herein, the LOQ refers to the lowest concentration above the LOD wherein the coefficient of variation (CV) of the measured concentrations less than about 20%.

In some cases, an assay method employed has a limit of detection and/or a limit of quantification of less than about or about 500 pg/mL, less than about or about 250 pg/mL, less than about or about 100 pg/mL, less than about or about 50 pg/mL, less than about or about 40 pg/mL, less than about or about 30 pg/mL, less than about or about 20 pg/mL, less than about or about 10 pg/mL, less than about or about 5 pg/mL, less than about or about 4 pg/mL, less than about or about 3 pg/mL, less than about or about 2 pg/mL, less than about or about 1 pg/mL, less than about or about 0.8 pg/mL, less than about or about 0.7 pg/mL, less than about or about 0.6 pg/mL, less than about or about 0.5 pg/mL, less than about or about 0.4 pg/mL, less than about or about 0.3 pg/mL, less than about or about 0.2 pg/mL, less than about or about 0.1 pg/mL, less than about or about 0.05 pg/mL, less than about or about 0.04 pg/mL, less than about or about 0.02 pg/mL, less than about or about 0.01 pg/mL, or less. In some cases, an assay method employed has a limit of quantification and/or a limit of detection between about 100 pg/mL and about 0.01 pg/mL, between about 50 pg/mL and about 0.02 pg/mL, between about 25 pg/mL and about 0.02 pg/mL, between about 10 pg/mL and about 0.02 pg/mL. As will be understood by those of ordinary skill the art, the LOQ and/or LOD may differ for each assay method and/or each biomarker determined with the same assay. In some embodiments, the LOD of an assay employed for detecting of beta-amyloid (e.g., beta-amyloid (1-42)) is about equal to or less than 0.02 pg/mL. In some embodiments, the LOQ for an assay employed for detecting beta-amyloid (e.g., beta-amyloid (1-42)) is equal to or less than 0.04 pg/mL.

In some embodiments, the concentration of biomarker molecules in the fluid sample that may be substantially accurately determined is less than about or about 5000 fM, less than about or about 3000 fM, less than about or about 2000 fM, less than about or about 1000 fM, less than about or about 500 fM, less than about or about 300 fM, less than about or about 200 fM, less than about or about 100 fM, less than about or about 50 fM, less than about or about 25 fM, less than about or about 10 fM, less than about or about 5 fM, less than about or about 2 fM, less than about or about 1 fM, less than about or about 0.5 fM, less than about or about 0.1 fM, or less. In some embodiments, the concentration of biomarker molecules in the fluid sample that may be substantially accurately determined is between about 5000 fM and about 0.1 fM, between about 3000 fM and about 0.1 fM, between about 1000 fM and about 0.1 fM, between about 1000 fM and about 1 fM, between about 100 fM and about 1 fM, between about 100 fM and about 0.1 fM, or the like. The concentration of analyte molecules or particles in a fluid sample may be considered to be substantially accurately determined if the measured concentration of the biomarker molecules in the fluid sample is within about 10% of the actual (e.g., true) concentration of the biomarker molecules in the fluid sample. In certain embodiments, the measured concentration of the biomarker molecules in the fluid sample may be within about 5%, within about 4%, within about 3%, within about 2%, within about 1%, within about 0.5%, within about 0.4%, within about 0.3%, within about 0.2% or within about 0.1%, of the actual concentration of the biomarker molecules in the fluid sample. In some cases, the measure of the concentration determined differs from the true (e.g., actual) concentration by no greater than about 20%, no greater than about 15%, no greater than 10%, no greater than 5%, no greater than 4%, no greater than 3%, no greater than 2%, no greater than 1%, or no greater than 0.5%. The accuracy of the assay method may be determined, in some embodiments, by determining the concentration of biomarker molecules in a fluid sample of a known concentration using the selected assay method.

In some embodiments, an assay method employs a step of spatially segregating biomarker molecules into a plurality of locations to facilitate detection/quantification, such that each location comprises/contains either zero or one or more biomarker molecules. Additionally, in some embodiments, the locations may be configured in a manner such that each location can be individually addressed. In some embodiments, a measure of the concentration of biomarker molecules in a fluid sample may be determined by detecting biomarker molecules immobilized with respect to a binding surface having affinity for at least one type of biomarker molecule. In certain embodiments the binding surface may form (e.g., a surface of a well/reaction vessel on a substrate) or be contained within (e.g., a surface of a capture object, such as a bead, contained within a well) one of a plurality of locations (e.g., a plurality of wells/reaction vessels) on a substrate (e.g., plate, dish, chip, optical fiber end, etc). At least a portion of the locations may be addressed and a measure indicative of the number/percentage/fraction of the locations containing at least one biomarker molecule may be made. In some cases, based upon the number/percentage/fraction, a measure of the concentration of biomarker molecules in the fluid sample may be determined. The measure of the concentration of biomarker molecules in the fluid sample may be determined by a digital analysis method/system optionally employing Poisson distribution adjustment and/or based at least in part on a measured intensity of a signal, as will be known to those of ordinary skill in the art. In some cases, the assay methods and/or systems may be automated.

Certain methods and systems which employ spatially segregating analyte molecules (e.g., biomarkers) are known in the art, and are described in U.S. Patent Application Publication No. US-2007-0259448 (Ser. No. 11/707,385), filed Feb. 16, 2007, entitled “METHODS AND ARRAYS FOR TARGET ANALYTE DETECTION AND DETERMINATION OF TARGET ANALYTE CONCENTRATION IN SOLUTION,” by Walt et al.; U.S. Patent Application Publication No. US-2007-0259385 (Serial No. 11/707,383), filed Feb. 16, 2007, entitled “METHODS AND ARRAYS FOR DETECTING CELLS AND CELLULAR COMPONENTS IN SMALL DEFINED VOLUMES,” by Walt et al.; U.S. Patent Application Publication No. US-2007-0259381 (Ser. No. 11/707,384), filed Feb. 16, 2007, entitled “METHODS AND ARRAYS FOR TARGET ANALYTE DETECTION AND DETERMINATION OF REACTION COMPONENTS THAT AFFECT A REACTION,” by Walt et al.; International Patent Publication No. WO 2009/029073 (International Patent Application No. PCT/US2007/019184), filed Aug. 30, 2007, entitled “METHODS OF DETERMINING THE CONCENTRATION OF AN ANALYTE IN SOLUTION,” by Walt et al.; U.S. Patent Application Publication No. US-2010-0075862 (Ser. No. 12/236,484), filed Sep. 23, 2008, entitled “HIGH SENSITIVITY DETERMINATION OF THE CONCENTRATION OF ANALYTE MOLECULES OR PARTICLES IN A FLUID SAMPLE,” by Duffy et al.; U.S. Patent Application Publication No. US-2010-00754072 (Ser. No. 12/236,486), filed Sep. 23, 2008, entitled “ULTRA-SENSITIVE DETECTION OF MOLECULES ON SINGLE MOLECULE ARRAYS,” by Duffy et al.; U.S. Patent Application Publication No. US-2010-0075439 (Ser. No. 12/236,488), filed Sep. 23, 2008, entitled “ULTRA-SENSITIVE DETECTION OF MOLECULES BY CAPTURE-AND-RELEASE USING REDUCING AGENTS FOLLOWED BY QUANTIFICATION,” by Duffy et al.; International Patent Publication No. WO2010/039179 (International Patent Application No. PCT/US2009/005248), filed Sep. 22, 2009, entitled “ULTRA-SENSITIVE DETECTION OF MOLECULES OR ENZYMES,” by Duffy et al.; U.S. Patent Application Publication No. US-2010-0075355 (Ser. No. 12/236,490), filed Sep. 23, 2008, entitled “ULTRA-SENSITIVE DETECTION OF ENZYMES BY CAPTURE-AND-RELEASE FOLLOWED BY QUANTIFICATION,” by Duffy et al .; U.S. patent application Ser. No. 12/731,130, filed Mar. 24, 2010, published as US-2011-0212848 on Sep. 1, 2011, entitled “ULTRA-SENSITIVE DETECTION OF MOLECULES OR PARTICLES USING BEADS OR OTHER CAPTURE OBJECTS,” by Duffy et al.; International Patent Application No. PCT/US2011/026645, filed Mar. 1, 2011, published as WO 2011/109364 on Sep. 9, 2011, entitled “ULTRA-SENSITIVE DETECTION OF MOLECULES OR PARTICLES USING BEADS OR OTHER CAPTURE OBJECTS,” by Duffy et al.; International Patent Application No. PCT/US2011/026657, filed Mar. 1, 2011, published as WO 2011/109372 on Sep. 9, 2011, entitled “ULTRA-SENSITIVE DETECTION OF MOLECULES USING DUAL DETECTION METHODS,” by Duffy et al.; U.S. patent application Ser. No. 12/731,135, filed Mar. 24, 2010, published as US-2011-0212462 on Sep. 1, 2011, entitled “ULTRA-SENSITIVE DETECTION OF MOLECULES USING DUAL DETECTION METHODS,” by Duffy et al.; International Patent Application No. PCT/US2011/026665, filed Mar. 1, 2011, published as WO 2011/109379 on Sep. 9, 2011, entitled “METHODS AND SYSTEMS FOR EXTENDING DYNAMIC RANGE IN ASSAYS FOR THE DETECTION OF MOLECULES OR PARTICLES,” by Rissin et al .; U.S. patent application Ser. No. 12/731,136, filed Mar. 24, 2010, published as US-2011-0212537 on Sep. 1, 2011, entitled “METHODS AND SYSTEMS FOR EXTENDING DYNAMIC RANGE IN ASSAYS FOR THE DETECTION OF MOLECULES OR PARTICLES,” by Duffy et al.; U.S. patent application Ser. No. 13/035,472, filed Feb. 25, 2011, entitled “SYSTEMS, DEVICES, AND METHODS FOR ULTRA-SENSITIVE DETECTION OF MOLECULES OR PARTICLES,” by Fournier et al.; U.S. patent application Ser. No. 13/037,987, filed Mar. 1, 2011, published as US-2011-0245097 on Oct. 6, 2011, entitled “METHODS AND SYSTEMS FOR EXTENDING DYNAMIC RANGE IN ASSAYS FOR THE DETECTION OF MOLECULES OR PARTICLES,” by Rissin et al.; each herein incorporated by reference.

Additional details of exemplary, non-limiting assay methods which comprise one or more steps of spatially segregating biomarker molecules will now be described. In certain embodiments, a method for detection and/or quantifying biomarker molecules in a sample comprises immobilizing a plurality of biomarker molecules with respect to a plurality of capture objects (e.g., beads) that each include a binding surface having affinity for at least one type of biomarker. For example, the capture objects may comprise a plurality of beads comprising a plurality of capture components (e.g., an antibody having specific affinity for a biomarker of interest, etc.). At least some of the capture objects (e.g., at least some associated with at least one biomarker molecule) may be spatially separated/segregated into a plurality of locations, and at least some of the locations may be addressed/interrogated (e.g., using an imaging system). A measure of the concentration of biomarker molecules in the fluid sample may be determined based on the information received when addressing the locations (e.g., using the information received from the imaging system and/or processed using a computer implemented control system). In some cases, a measure of the concentration may be based at least in part on the number of locations determined to contain a capture object that is or was associated with at least one biomarker molecule. In other cases and/or under differing conditions, a measure of the concentration may be based at least in part on an intensity level of at least one signal indicative of the presence of a plurality of biomarker molecules and/or capture objects associated with a biomarker molecule at one or more of the addressed locations.

In some embodiments, the number/percentage/fraction of locations containing a capture object but not containing a biomarker molecule may also be determined and/or the number/percentage/fraction of locations not containing any capture object may also be determined. In such embodiments, a measure of the concentration of biomarker molecules in the fluid sample may be based at least in part on the ratio of the number of locations determined to contain a capture object associated with a biomarker molecule to the total number of locations determined to contain a capture object not associated with a biomarker molecule, and/or a measure of the concentration of biomarker molecule in the fluid sample may be based at least in part on the ratio of the number of locations determined to contain a capture object associated with a biomarker molecule to the number of locations determined to not contain any capture objects, and/or a measure of the concentration of biomarker molecule in the fluid sample may be based at least in part on the ratio of the number of locations determined to contain a capture object associated with a biomarker molecule to the number of locations determined to contain a capture object.

In yet other embodiments, a measure of the concentration of biomarker molecules in a fluid sample may be based at least in part on the ratio of the number of locations determined to contain a capture object and a biomarker molecule to the total number of locations addressed and/or analyzed.

In certain embodiments, at least some of the plurality of capture objects (e.g., at least some associated with at least one biomarker molecule) are spatially separated into a plurality of locations, for example, a plurality of reaction vessels in an array format. The plurality of reaction vessels may be formed in, on and/or of any suitable material, and in some cases, the reaction vessels can be sealed or may be formed upon the mating of a substrate with a scaling component, as discussed in more detail below. In certain embodiments, especially where quantization of the capture objects associated with at least one biomarker molecule is desired, the partitioning of the capture objects can be performed such that at least some (e.g., a statistically significant fraction; e.g., as described in International Patent Application No. PCT/US2011/026645, filed Mar. 1, 2011, published as WO 2011/109364 on Sep. 9, 2011, entitled “ULTRA-SENSITIVE DETECTION OF MOLECULES OR PARTICLES USING BEADS OR OTHER CAPTURE OBJECTS,” by Duffy et al.) of the reaction vessels comprise at least one or, in certain cases, only one capture object associated with at least one biomarker molecule and at least some (e.g., a statistically significant fraction) of the reaction vessels comprise an capture object not associated with any biomarker molecules. The capture objects associated with at least one biomarker molecule may be quantified in certain embodiments, thereby allowing for the detection and/or quantification of biomarker molecules in the fluid sample by techniques described in more detail herein.

An exemplary assay method may proceed as follows. A sample fluid containing or suspected of containing biomarker molecules is provided. An assay consumable comprising a plurality of assay sites is exposed to the sample fluid. In some cases, the biomarker molecules are provided in a manner (e.g., at a concentration) such that a statistically significant fraction of the assay sites contain a single biomarker molecule and a statistically significant fraction of the assay sites do not contain any biomarker molecules. The assay sites may optionally be exposed to a variety of reagents (e.g., using a reagent loader) and or rinsed. The assay sites may then optionally be sealed and imaged (see, for example, U.S. patent application Ser. No. 13/035,472, filed Feb. 25, 2011, entitled “SYSTEMS, DEVICES, AND METHODS FOR ULTRA-SENSITIVE DETECTION OF MOLECULES OR PARTICLES,” by Fournier et al.). The images are then analyzed (e.g., using a computer implemented control system) such that a measure of the concentration of the biomarker molecules in the fluid sample may be obtained, based at least in part, by determination of the number/fraction/percentage of assay sites which contain a biomarker molecule and/or the number/fraction/percentage of sites which do not contain any biomarkers molecules. In some cases, the biomarker molecules are provided in a manner (e.g., at a concentration) such that at least some assay sites comprise more than one biomarker molecule. In such embodiments, a measure of the concentration of biomarker molecules in the fluid sample may be obtained at least in part on an intensity level of at least one signal indicative of the presence of a plurality of biomarkers molecules at one or more of the assay sites.

In some cases, the methods optionally comprise exposing the fluid sample to a plurality of capture objects, for example, beads. At least some of the biomarker molecules are immobilized with respect to a bead. In some cases, the biomarker molecules are provided in a manner (e.g., at a concentration) such that a statistically significant fraction of the beads associate with a single biomarker molecule and a statistically significant fraction of the beads do not associate with any biomarker molecules. At least some of the plurality of beads (e.g., those associated with a single biomarker molecule or not associated with any biomarker molecules) may then be spatially separated/segregated into a plurality of assay sites (e.g., of an assay consumable). The assay sites may optionally be exposed to a variety of reagents and/or rinsed. At least some of the assay sites may then be addressed to determine the number of assay sites containing a biomarker molecule. In some cases, the number of assay sites containing a bead not associated with a biomarker molecule, the number of assay sites not containing a bead and/or the total number of assay sites addressed may also be determined. Such determination(s) may then be used to determine a measure of the concentration of biomarker molecules in the fluid sample. In some cases, more than one biomarker molecule may associate with a bead and/or more than one bead may be present in an assay site. In some cases, the plurality biomarker molecules may be exposed to at least one additional reaction component prior to, concurrent with, and/or following spatially separating at least some of the biomarker molecules into a plurality of locations.

The biomarker molecules may be directly detected or indirectly detected. In the case of direct detection, a biomarker molecule may comprise a molecule or moiety that may be directly interrogated and/or detected (e.g., a fluorescent entity). In the case of indirect detection, an additional component is used for determining the presence of the biomarker molecule. For example, the biomarker molecules (e.g., optionally associated with a bead) may be exposed to at least one type of binding ligand. A “binding ligand,” is any molecule, particle, or the like which specifically binds to or otherwise specifically associates with a biomarker molecule to aid in the detection of the biomarker molecule. In certain embodiments, a binding ligand may be adapted to be directly detected (e.g., the binding ligand comprises a detectable molecule or moiety) or may be adapted to be indirectly detected (e.g., including a component that can convert a precursor labeling agent into a labeling agent). A component of a binding ligand may be adapted to be directly detected in embodiments where the component comprises a measurable property (e.g., a fluorescence emission, a color, etc.). A component of a binding ligand may facilitate indirect detection, for example, by converting a precursor labeling agent into a labeling agent (e.g., an agent that is detected in an assay). A “precursor labeling agent” is any molecule, particle, or the like, that can be converted to a labeling agent upon exposure to a suitable converting agent (e.g., an enzymatic component). A “labeling agent” is any molecule, particle, or the like, that facilitates detection, by acting as the detected entity, using a chosen detection technique. In some embodiments, the binding ligand may comprise an enzymatic component (e.g., horseradish peroxidase, beta-galactosidase, alkaline phosphatase, etc.). A first type of binding ligand may or may not be used in conjunction with additional binding ligands (e.g., second type, etc.).

More than one type of binding may be employed in any given assay method, for example, a first type of binding ligand and a second type of binding ligand. In one example, the first type of binding ligand is able to associate with a first type of biomarker molecule and the second type of binding ligand is able to associate with the first binding ligand. In another example, both a first type of binding ligand and a second type of binding ligand may associate with the same or different epitopes of a single biomarker molecule, as described herein. In some embodiments, at least one binding ligand comprises an enzymatic component.

In some embodiments, a binding ligand and/or a biomarker may comprise an enzymatic component. The enzymatic component may convert a precursor labeling agent (e.g., an enzymatic substrate) into a labeling agent (e.g., a detectable product). A measure of the concentration of biomarker molecules in the fluid sample can then be determined based at least in part by determining the number of locations containing a labeling agent (e.g., by relating the number of locations containing a labeling agent to the number of locations containing a biomarker molecule (or number of capture objects associated with at least one biomarker molecule to total number of capture objects)). Non-limiting examples of enzymes or enzymatic components include horseradish peroxidase, beta-galactosidase, and alkaline phosphatase. Other non-limiting examples of systems or methods for detection include embodiments where nucleic acid precursors are replicated into multiple copies or converted to a nucleic acid that can be detected readily, such as the polymerase chain reaction (PCR), rolling circle amplification (RCA), ligation, Loop-Mediated Isothermal Amplification (LAMP), etc. Such systems and methods will be known to those of ordinary skill in the art, for example, as described in “DNA Amplification: Current Technologies and Applications,” Vadim Demidov et al., 2004.

Another exemplary embodiment of indirect detection is as follows. In some cases, the biomarker molecules may be exposed to a precursor labeling agent (e.g., enzymatic substrate) and the enzymatic substrate may be converted to a detectable product (e.g., fluorescent molecule) upon exposure to a biomarker molecule.

The assay methods and systems may employ a variety of different components, steps, and/or other aspects that will be known and understood by those of ordinary skill in the art. For example, a method may further comprise determining at least one background signal determination (e.g., and further comprising subtracting the background signal from other determinations), wash steps, and the like. In some cases, the assays or systems may include the use of at least one binding ligand, as described herein. In some cases, the measure of the concentration of biomarker molecules in a fluid sample is based at least in part on comparison of a measured parameter to a calibration curve. In some instances, the calibration curve is formed at least in part by determination at least one calibration factor, as described above.

In certain embodiments, solubilized, or suspended precursor labeling agents may be employed, wherein the precursor labeling agents are converted to labeling agents which are insoluble in the liquid and/or which become immobilized within/near the location (e.g., within the reaction vessel in which the labeling agent is formed). Such precursor labeling agents and labeling agents and their use is described in commonly owned U.S. Patent Application Publication No. US-2010-0075862 (Ser. No. 12/236,484), filed Sep. 23, 2008, entitled “HIGH SENSITIVITY DETERMINATION OF THE CONCENTRATION OF ANALYTE MOLECULES OR PARTICLES IN A FLUID SAMPLE,” by Duffy et al., incorporated herein by reference.

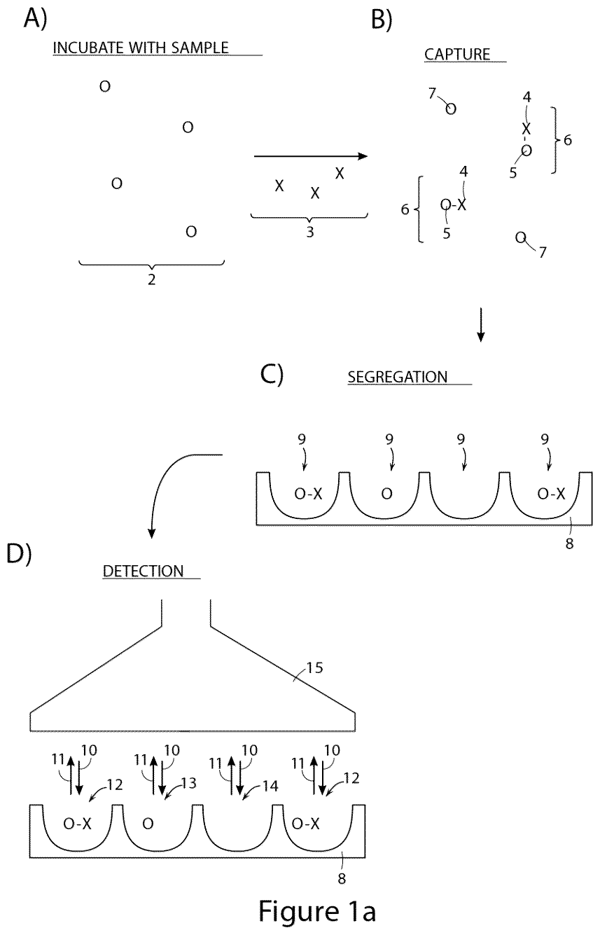

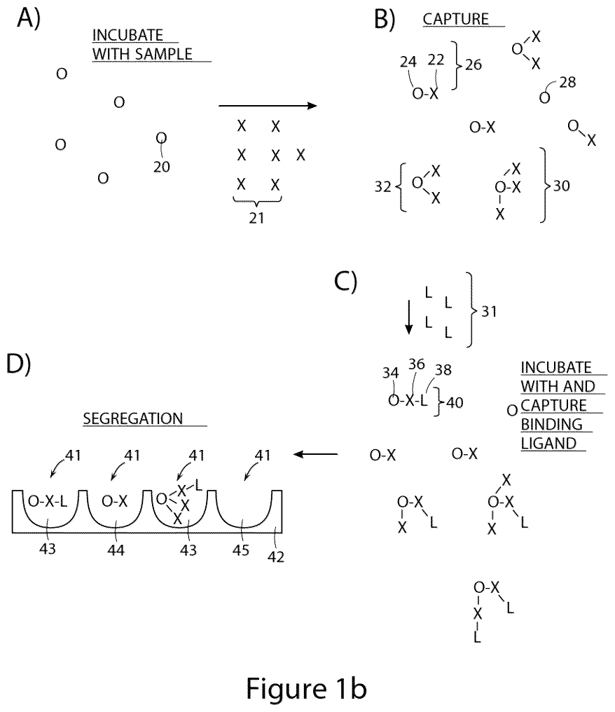

An exemplary embodiment of an assay method that may be used in certain embodiments of the invention is illustrated in FIG. 1a. A plurality of capture objects 2, are provided (step (A)). In this particular example, the plurality of capture objects comprises a plurality of beads. The beads are exposed to a fluid sample containing a plurality of biomarker molecules 3 (e.g., beads 2 are incubated with biomarker molecules 3). At least some of the biomarker molecules are immobilized with respect to a bead. In this example, the biomarker molecules are provided in a manner (e.g., at a concentration) such that a statistically significant fraction of the beads associate with a single biomarker molecule and a statistically significant fraction of the beads do not associate with any biomarker molecules. For example, as shown in step (B), biomarker molecule 4 is immobilized with respect to bead 5, thereby forming complex 6, whereas some beads 7 are not associated with any biomarker molecules. It should be understood, in some embodiments, more than one biomarker molecule may associate with at least some of the beads, as described herein. At least some of the plurality of beads (e.g., those associated with a single biomarker molecule or not associated with any biomarker molecules) may then be spatially separated/segregated into a plurality of locations. As shown in step (C), the plurality of locations is illustrated as substrate 8 comprising a plurality of wells/reaction vessels 9. In this example, each reaction vessel comprises either zero or one beads. At least some of the reaction vessels may then be addressed (e.g., optically or via other detection means) to determine the number of locations containing a biomarker molecule. For example, as shown in step (D), the plurality of reaction vessels are interrogated optically using light source 15, wherein each reaction vessel is exposed to electromagnetic radiation (represented by arrows 10) from light source 15. The light emitted (represented by arrows 11) from each reaction vessel is determined (and/or recorded) by detector 15 (in this example, housed in the same system as light source 15). The number of reaction vessels containing a biomarker molecule (e.g., reaction vessels 12) is determined based on the light detected from the reaction vessels. In some cases, the number of reaction vessels containing a bead not associated with a biomarker molecule (e.g., reaction vessel 13), the number of wells not containing a bead (e.g., reaction vessel 14) and/or the total number of wells addressed may also be determined. Such determination(s) may then be used to determine a measure of the concentration of biomarker molecules in the fluid sample.

A non-limiting example of an embodiment where a capture object is associated with more than one biomarker molecule is illustrated in FIG. 1b. A plurality of capture objects 20 are provided (step (A)). In this example, the plurality of capture objects comprises a plurality of beads. The plurality of beads is exposed to a fluid sample containing plurality of biomarker molecules 21 (e.g., beads 20 are incubated with biomarker molecules 21). At least some of the biomarker molecules are immobilized with respect to a bead. For example, as shown in step (B), biomarker molecule 22 is immobilized with respect to bead 24, thereby forming complex 26. Also illustrated is complex 30 comprising a bead immobilized with respect to three biomarker molecules and complex 32 comprising a bead immobilized with respect to two biomarker molecules. Additionally, in some cases, some of the beads may not associate with any biomarker molecules (e.g., bead 28). The plurality of beads from step (B) is exposed to a plurality of binding ligands 31. As shown in step (C), a binding ligand associates with some of the biomarker molecules immobilized with respect to a bead. For example, complex 40 comprises bead 34, biomarker molecule 36, and binding ligand 38. The binding ligands are provided in a manner such that a statistically significant fraction of the beads comprising at least one biomarker molecule become associated with at least one binding ligand (e.g., one, two, three, etc.) and a statistically significant fraction of the beads comprising at least one biomarker molecule do not become associated with any binding ligands. At least a portion of the plurality of beads from step (C) are then spatially separated into a plurality of locations. As shown in step (D), in this example, the locations comprise a plurality of reaction vessels 41 on a substrate 42. The plurality of reaction vessels may be exposed to the plurality of beads from step (C) such at each reaction vessel contains zero or one beads. The substrate may then be analyzed to determine the number of reaction vessels containing a binding ligand (e.g., reaction vessels 43), wherein in the number may be related to a measure of the concentration of biomarker molecules in the fluid sample. In some cases, the number of reaction vessels containing a bead and not containing a binding ligand (e.g., reaction vessel 44), the number of reaction vessels not containing a bead (e.g., reaction vessel 45), and/or the total number of reaction vessels addressed/analyzed may also be determined. Such determination(s) may then be used to determine a measure of the concentration of biomarker molecules in the fluid sample.

In some embodiments, a plurality of locations may be addressed and/or a plurality of capture objects and/or species/molecules/particles of interest may be detected substantially simultaneously. “Substantially simultaneously” when used in this context, refers to addressing/detection of the locations/capture objects/species/molecules/particles of interest at approximately the same time such that the time periods during which at least two locations/capture objects/species/molecules/particles of interest are addressed/detected overlap, as opposed to being sequentially addressed/detected, where they would not. Simultaneous addressing/detection can be accomplished by using various techniques, including optical techniques (e.g., CCD detector). Spatially segregating capture objects/species/molecules/particles into a plurality of discrete, resolvable locations, according to some embodiments facilitates substantially simultaneous detection by allowing multiple locations to be addressed substantially simultaneously. For example, for embodiments where individual species/molecules/particles are associated with capture objects that are spatially segregated with respect to the other capture objects into a plurality of discrete, separately resolvable locations during detection, substantially simultaneously addressing the plurality of discrete, separately resolvable locations permits individual capture objects, and thus individual species/molecules/particles (e.g., biomarker molecules) to be resolved. For example, in certain embodiments, individual molecules/particles of a plurality of molecules/particles are partitioned across a plurality of reaction vessels such that each reaction vessel contains zero or only one species/molecule/particle. In some cases, at least about 80%, at least about 85%, at least about 90%, at least about 95%, at least about 96%, at least about 97%, at least about 98%, at least about 99%, at least about 99.5% of all species/molecules/particles are spatially separated with respect to other species/molecules/particles during detection. A plurality of species/molecules/particles may be detected substantially simultaneously within a time period of less than about 1 second, less than about 500 milliseconds, less than about 100 milliseconds, less than about 50 milliseconds, less than about 10 milliseconds, less than about 1 millisecond, less than about 500 microseconds, less than about 100 microseconds, less than about 50 microseconds, less than about 10 microseconds, less than about 1 microsecond, less than about 0.5 microseconds, less than about 0.1 microseconds, or less than about 0.01 microseconds, less than about 0.001 microseconds, or less. In some embodiments, the plurality of species/molecules/particles may be detected substantially simultaneously within a time period of between about 100 microseconds and about 0.001 microseconds, between about 10 microseconds and about 0.01 microseconds, or less.

In some embodiments, the locations are optically interrogated. The locations exhibiting changes in their optical signature may be identified by a conventional optical train and optical detection system. Depending on the detected species (e.g., type of fluorescence entity, etc.) and the operative wavelengths, optical filters designed for a particular wavelength may be employed for optical interrogation of the locations. In embodiments where optical interrogation is used, the system may comprise more than one light source and/or a plurality of filters to adjust the wavelength and/or intensity of the light source. In some embodiments, the optical signal from a plurality of locations is captured using a CCD camera.

In some embodiments of the present invention, the plurality of reaction vessels may be scaled (e.g., after the introduction of the biomarker molecules, binding ligands, and/or precursor labeling agent), for example, through the mating of the second substrate and a sealing component. The sealing of the reaction vessels may be such that the contents of each reaction vessel cannot escape the reaction vessel during the remainder of the assay. In some cases, the reaction vessels may be sealed after the addition of the biomarker molecules and, optionally, at least one type of precursor labeling agent to facilitate detection of the biomarker molecules. For embodiments employing precursor labeling agents, by sealing the contents in some or each reaction vessel, a reaction to produce the detectable labeling agents can proceed within the sealed reaction vessels, thereby producing a detectable amount of labeling agents that is retained in the reaction vessel for detection purposes.

The plurality of locations may be formed may be formed using a variety of methods and/or materials. In some embodiments, the plurality of locations comprises a plurality of reaction vessels/wells on a substrate. In some cases, the plurality of reaction vessels is formed as an array of depressions on a first surface. In other cases, however, the plurality of reaction vessels may be formed by mating a scaling component comprising a plurality of depressions with a substrate that may either have a featureless surface or include depressions aligned with those on the sealing component. Any of the device components, for example, the substrate or sealing component, may be fabricated from a compliant material, e.g., an elastomeric polymer material, to aid in sealing. The surfaces may be or made to be hydrophobic or contain hydrophobic regions to minimize leakage of aqueous samples from the microwells. The reactions vessels, in certain embodiments, may be configured to receive and contain only a single capture object.

In some embodiments, the reaction vessels may all have approximately the same volume. In other embodiments, the reaction vessels may have differing volumes. The volume of each individual reaction vessel may be selected to be appropriate to facilitate any particular assay protocol. For example, in one set of embodiments where it is desirable to limit the number of capture objects used for biomarker capture contained in each vessel to a small number, the volume of the reaction vessels may range from attoliters or smaller to nanoliters or larger depending upon the nature of the capture objects, the detection technique and equipment employed, the number and density of the wells on the substrate and the expected concentration of capture objects in the fluid applied to the substrate containing the wells. In one embodiment, the size of the reaction vessel may be selected such only a single capture object used for biomarker capture can be fully contained within the reaction vessel (see, for example, U.S. patent application Ser. No. 12/731,130, filed Mar. 24, 2010, published as US-2011-0212848 on Sep. 1, 2011, entitled “ULTRA-SENSITIVE DETECTION OF MOLECULES OR PARTICLES USING BEADS OR OTHER CAPTURE OBJECTS,” by Duffy et al.; International Patent Application No. PCT/US2011/026645, filed Mar. 1, 2011, published as WO 2011/109364 on Sep. 9, 2011, entitled “ULTRA-SENSITIVE DETECTION OF MOLECULES OR PARTICLES USING BEADS OR OTHER CAPTURE OBJECTS,” by Duffy et al., each herein incorporated by reference).

In some embodiments, the reaction vessels may have a volume between about 1 femtoliter and about 1 picoliter, between about 1 femtoliters and about 100 femtoliters, between about 10 attoliters and about 100 picoliters, between about 1 picoliter and about 100 picoliters, between about 1 femtoliter and about 1 picoliter, or between about 30 femtoliters and about 60 femtoliters. In some cases, the reaction vessels have a volume of less than about 1 picoliter, less than about 500 femtoliters, less than about 100 femtoliters, less than about 50 femtoliters, or less than about 1 femtoliter. In some cases, the reaction vessels have a volume of about 10 femtoliters, about 20 femtoliters, about 30 femtoliters, about 40 femtoliters, about 50 femtoliters, about 60 femtoliters, about 70 femtoliters, about 80 femtoliters, about 90 femtoliters, or about 100 femtoliters.

The total number of locations and/or density of the locations employed in an assay (e.g., the number/density of reaction vessels in an array) can depend on the composition and end use of the array. For example, the number of reaction vessels employed may depend on the number of types of biomarker molecule and/or binding ligand employed, the suspected concentration range of the assay, the method of detection, the size of the capture objects, the type of detection entity (e.g., free labeling agent in solution, precipitating labeling agent, etc.). Arrays containing from about 2 to many billions of reaction vessels (or total number of reaction vessels) can be made by utilizing a variety of techniques and materials. Increasing the number of reaction vessels in the array can be used to increase the dynamic range of an assay or to allow multiple samples or multiple types of biomarker molecules to be assayed in parallel. The array may comprise between one thousand and one million reaction vessels per sample to be analyzed. In some cases, the array comprises greater than one million reaction vessels. In some embodiments, the array comprises between about 1,000 and about 50,000, between about 1,000 and about 1,000,000, between about 1,000 and about 10,000, between about 10,000 and about 100,000, between about 100,000 and about 1,000,000, between about 100,000 and about 500,000, between about 1,000 and about 100,000, between about 50,000 and about 100,000, between about 20,000 and about 80,000, between about 30,000 and about 70,000, between about 40,000 and about 60,000 reaction vessels. In some embodiments, the array comprises about 10,000, about 20,000, about 50,000, about 100,000, about 150,000, about 200,000, about 300,000, about 500,000, about 1,000,000, or more, reaction vessels.

The array of reaction vessels may be arranged on a substantially planar surface or in a non-planar three-dimensional arrangement. The reaction vessels may be arrayed in a regular pattern or may be randomly distributed. In a specific embodiment, the array is a regular pattern of sites on a substantially planar surface permitting the sites to be addressed in the X-Y coordinate plane.

In some embodiments, the reaction vessels are formed in a solid material. As will be appreciated by those in the art, the number of potentially suitable materials in which the reaction vessels can be formed is very large, and includes, but is not limited to, glass (including modified and/or functionalized glass), plastics (including acrylics, polystyrene and copolymers of styrene and other materials, polypropylene, polyethylene, polybutylene, polyurethanes, cyclic olefin copolymer (COC), cyclic olefin polymer (COP), Teflon®, polysaccharides, nylon or nitrocellulose, etc.), elastomers (such as poly(dimethyl siloxane) and poly urethanes), composite materials, ceramics, silica or silica-based materials (including silicon and modified silicon), carbon, metals, optical fiber bundles, or the like. In general, the substrate material may be selected to allow for optical detection without appreciable autofluorescence. In certain embodiments, the reaction vessels may be formed in a flexible material.

A reaction vessel in a surface (e.g., substrate or sealing component) may be formed using a variety of techniques known in the art, including, but not limited to, photolithography, stamping techniques, molding techniques, etching techniques, or the like. As will be appreciated by those of the ordinary skill in the art, the technique used can depend on the composition and shape of the supporting material and the size and number of reaction vessels.

In a particular embodiment, an array of reaction vessels is formed by creating microwells on one end of a fiber optic bundle and utilizing a planar compliant surface as a sealing component. In certain such embodiments, an array of reaction vessels in the end of a fiber optic bundle may be formed as follows. First, an array of microwells is etched into the end of a polished fiber optic bundle. Techniques and materials for forming and etching a fiber optic bundle are known to those of ordinary skill in the art. For example, the diameter of the optical fibers, the presence, size and composition of core and cladding regions of the fiber, and the depth and specificity of the etch may be varied by the etching technique chosen so that microwells of the desired volume may be formed. In certain embodiments, the etching process creates microwells by preferentially etching the core material of the individual glass fibers in the bundle such that each well is approximately aligned with a single fiber and isolated from adjacent wells by the cladding material. Potential advantages of the fiber optic array format is that it can produce thousands to millions of reaction vessels without complicated microfabrication procedures and that it can provide the ability to observe and optically address many reaction vessels simultaneously.

Each microwell may be aligned with an optical fiber in the bundle so that the fiber optic bundle can carry both excitation and emission light to and from the wells, enabling remote interrogation of the well contents. Further, an array of optical fibers may provide the capability for simultaneous or non-simultaneous excitation of molecules in adjacent vessels, without signal “cross-talk” between fibers. That is, excitation light transmitted in one fiber does not escape to a neighboring fiber.

Alternatively, the equivalent structures of a plurality of reaction vessels may be fabricated using other methods and materials that do not utilize the ends of an optical fiber bundle as a substrate. For example, the array may be a spotted, printed or photolithographically fabricated substrate produced by techniques known in the art; see for example WO95/25116; WO95/35505; PCT US98/09163; U.S. Pat. Nos. 5,700,637, 5,807,522, 5,445,934, 6,406,845, and 6,482,593. In some cases, the array may be produced using molding, embossing, and/or etching techniques as will be known to those of ordinary skill in the art.

In some embodiments, the plurality of locations may not comprise a plurality of reaction vessels/wells. For example, in embodiments where capture objects are employed, a patterned substantially planar surface may be employed and the patterned areas form a plurality of locations. In some cases, the patterned areas may comprise substantially hydrophilic surfaces which are substantially surrounded by substantially hydrophobic surfaces. In certain embodiments, a plurality of capture objects (e.g., beads) may be substantially surrounded by a substantially hydrophilic medium (e.g., comprising water), and the beads may be exposed to the patterned surface such that the beads associate in the patterned areas (e.g., the hydrophilic locations on the surface), thereby spatially segregating the plurality of beads. For example, in one such embodiment, a substrate may be or include a gel or other material able to provide a sufficient barrier to mass transport (e.g., convective and/or diffusional barrier) to prevent capture objects used for biomarker capture and/or precursor labeling agent and/or labeling agent from moving from one location on or in the material to another location so as to cause interference or cross-talk between spatial locations containing different capture objects during the time frame required to address the locations and complete the assay. For example, in one embodiment, a plurality of capture objects is spatially separated by dispersing the capture objects on and/or in a hydrogel material. In some cases, a precursor labeling agent may be already present in the hydrogel, thereby facilitating development of a local concentration of the labeling agent (e.g., upon exposure to a binding ligand or biomarker molecule carrying an enzymatic component). As still yet another embodiment, the capture objects may be confined in one or more capillaries. In some cases, the plurality of capture objects may be absorbed or localized on a porous or fibrous substrate, for example, filter paper. In some embodiments, the capture objects may be spatially segregated on a uniform surface (e.g., a planar surface), and the capture objects may be detected using precursor labeling agents which are converted to substantially insoluble or precipitating labeling agents that remain localized at or near the location of where the corresponding capture object is localized. The use of such substantially insoluble or precipitating labeling agents is described herein. In some cases, single biomarker molecules may be spatially segregated into a plurality of droplets. That is, single biomarker molecules may be substantially contained in a droplet containing a first fluid. The droplet may be substantially surrounded by a second fluid, wherein the second fluid is substantially immiscible with the first fluid.

In some embodiments, during the assay, at least one washing step may be carried out. In certain embodiments, the wash solution is selected so that it does not cause appreciable change to the configuration of the capture objects and/or biomarker molecules and/or does not disrupt any specific binding interaction between at least two components of the assay (e.g., a capture component and a biomarker molecule). In other cases, the wash solution may be a solution that is selected to chemically interact with one or more assay components. As will be understood by those of ordinary skill in the art, a wash step may be performed at any appropriate time point during the inventive methods. For example, a plurality of capture objects may be washed after exposing the capture objects to one or more solutions comprising biomarker molecules, binding ligands, precursor labeling agents, or the like. As another example, following immobilization of the biomarker molecules with respect to a plurality of capture objects, the plurality of capture objects may be subjected to a washing step thereby removing any biomarker molecules not specifically immobilized with respect to a capture object.

Other assay methods in addition to those described herein are known in the art and may be used in connection with the inventive methods. For example, various analyzers are commercially available for the determination of the concentration of biomarkers. The assay methods employed should meet the algorithm requirements for LOD and LOQ.

The following examples are included to demonstrate various features of the invention. Those of ordinary skill in the art should, in light of the present disclosure, will appreciate that many changes can be made in the specific embodiments which are disclosed while still obtaining a like or similar result without departing from the scope of the invention as defined by the appended claims. Accordingly, the following examples are intended only to illustrate certain features of the present invention, but do not necessarily exemplify the full scope of the invention.

### Example 1

The following example provides experimental details relating to prognostication of neurological outcome in comatose survivors of oxygen deprivation.

26 unconscious patients were resuscitated with restoration of spontaneous circulation (ROSC). All included patients were >18 years old, had systolic blood pressure >80 mmHg for more than 5 min after ROSC, and were unconscious (prior to the use of sedatives/hypnotics) with a Glasgow Coma Scale (GCS) ≤7. Patients with a terminal disease or a primary coagulopathy were excluded, as were patients admitted more than 6 h after cardiac arrest. Serial blood samples were collected within 6 h after cardiac arrest, and continued at intervals from 1-108 h. Inclusion criteria included age >18 years old, systolic BP >80 mmHg after ROSC, and a Glasgow Coma Scale ≤7.

Upon admission, hypothermia treatment was started immediately after resuscitation. Ventilation was administered during the coma period, with a target PaO₂ of ≥12 kPa (90 mmHg) and PaCO₂ between 5.0 and 5.5 kPa (38-41 mmHg). Targeted mean arterial pressure was 65-100 mmHg, with application of inotropic/vasopressor support, if required, using dobutamine as the first line medication, followed by noradrenaline (norepinephrine) or adrenaline (epinephrine), if necessary. If the patient was considered euvolaemic but had a diuresis of less than 0.5 ml/kg/h, furosemide was administered. Furosemide was also given if the intensive care physician considered that the patient had a fluid overload. All patients received an arterial line in the radial or femoral artery for blood sampling.

Patient outcome was assessed in accordance with the Glasgow-Pittsburgh cerebral performance category (CPC) scale at discharge from the intensive care unit and 6 months later. The CPC scale ranges from 1 to 5, with 1 representing mildest possible neurological deficit (patient is able to return to work), and 4-5 representing the most severe deficit (vegetative) and death. A CPC of 1 or 2 was considered a “good” outcome and a CPC score of 3-5 a “poor” outcome. For patients who died after ICU discharge, the better of the two scores was used, as recommended by the Utstein templates.

Serial blood samples were collected for each sample, starting as soon as possible in the emergency phase (within 6 h after cardiac arrest), and continuing at 1, 2, 6, 12, 24, 48, 72, 96, and 108 h after cardiac arrest. Serum aliquots were frozen until assayed. Samples were measured in triplicate using the assay method described in Example 2. The assay had a dynamic range to 250 pg/mL, with limit of quantification of less than 0.04 pg/mL, enabling measurement of low abundance serum Aβ42 (i.e., beta-amyloid (1-42)) in all samples. Applicant notes the measurements carried out in this study would not be possible with most current Aβ42 assay methods due to insufficient sensitivity. All patients exhibited a significant time-dependent elevation of Aβ42. After a lag period of about 10 or more hours, significant Aβ42 elevations were observed. Elevations ranged from approximately 50% to over 30-fold, with most elevations in the range of 3-10-fold (average approximately 7-fold).

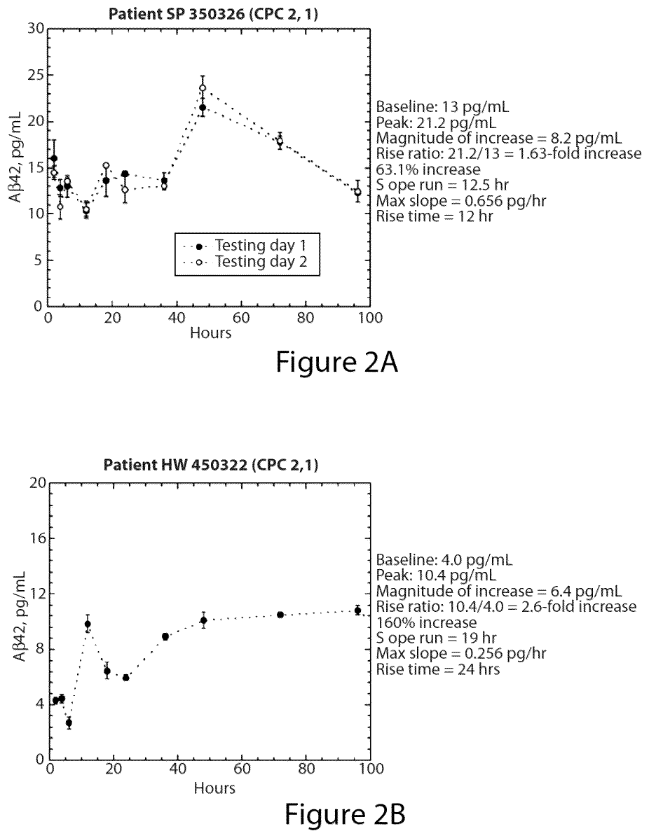

FIG. 2 depicts representative exemplary time course profiles for patients with (a-c) “good” and (d-f) “poor” 6-month outcomes. Serum Aβ42 measurements followed resuscitation from cardiac arrest. CPC scores depicted are after discharge from the ICU and 6 months later. In particular, FIG. 2 (a) depicts data from the patient exhibiting the highest baseline Aβ42 and smallest relative increase in Aβ42 among all patients (two sets of data points run on different days are shown); (d) depicts data from the patient exhibiting the largest relative increase from baseline (35-fold), with Aβ42 rising 60+ hours; (c and f) depict comparative data from two patients with a CPC score of 3 upon discharge from the ICU. The patient exhibiting the more modest increase in Aβ42 (c) recovered good brain function (CPC 1), while the patient exhibiting the greater increase in Aβ42 (f) did not recover.

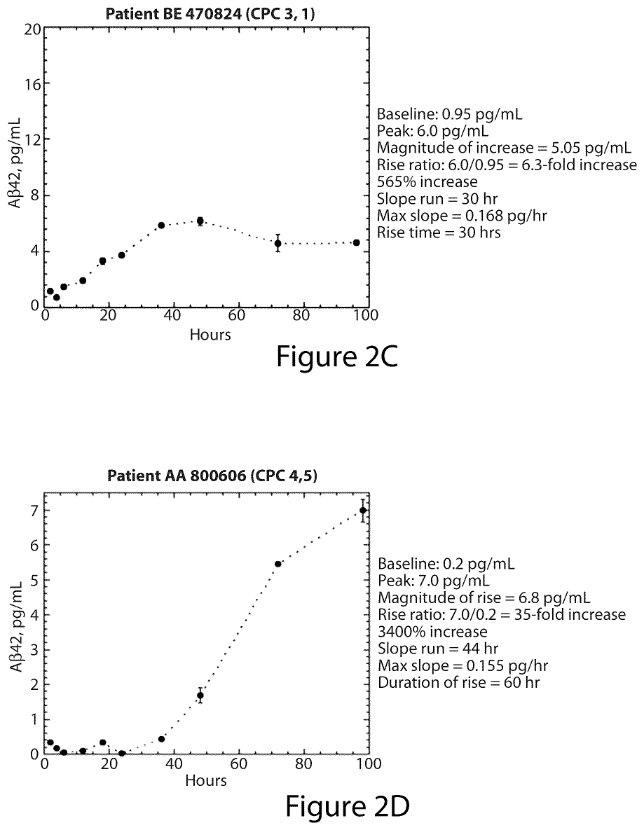

Quantitative differences in the appearance of the Aβ42 profiles were examined to determine a correlation with patient outcome. Profile parameters examined were: increase in beta-amyloid concentration (e.g., the magnitude of the rise), the fold increase of beta-amyloid concentration (e.g., the ratio of the peak Aβ42 to the baseline value), the maximum slope of the Aβ42 rise, and the duration of increase of beta-amyloid concentration (e.g., duration of over which the Aβ42 exhibited increasing values) (see FIG. 2 for an exemplary graph including these parameters). Table 2 shows the CPC assessment for the patients by their attending neurologists. Table 3 shows the correlation of Aβ42 profile parameters (FIG. 3) with overall six-month outcome (0=“good,” 1=“poor”). For each Aβ42 profile parameter, patients were sorted in either “good” or “poor” columns. Average values of each parameter for good outcomes were compared with average values for poor outcomes. Student t-test results for significance between good vs. poor were then calculated, as summarized in Table 3. In addition, an Aβ42 Score was calculated by summing the Aβ42 profile parameters of magnitude, rise ratio, and duration of rise (the three parameters that demonstrated statistical significance). Aβ42 Score was also evaluated for statistical significance between good and poor outcomes, and its significance was found to be enhanced relative to the individual Aβ42 profile parameters (P=0.00009). Table 4 summarizes the statistical significance of Aβ42 profile parameters and Aβ42 Score. A P of 0.00009 indicates a high degree of statistical significance that Aβ42 Score is different between good and poor outcomes. 100% of patients with an Aβ42 Score >50 had poor six month outcomes. 90% of patients with an Aβ42 Score <50 had good six month outcomes.

These results indicate that blood tests for Aβ42 in the initial days following hypoxic insult can predict six-month patient outcome with a high degree of accuracy.

TABLE 2

| | | | | [symbol] |
|---|---|---|---|---|
| | | | | [symbol] |
| | | CPC ICU | CPC 6 | [symbol] |
| [symbol] | | [symbol] | [symbol] | [symbol] |
| 1 | [symbol] | 1 | 1 | 0 |
| 2 | [symbol] | 2 | 1 | 0 |
| 3 | [symbol] | 1 | 1 | 0 |
| 4 | [symbol] | 1 | 1 | 0 |
| 5 | [symbol] | 3 | 1 | 0 |
| 6 | [symbol] | 5 | 5 | 1 |
| 7 | [symbol] | 4 | 5 | 1 |
| 8 | [symbol] | 4 | 2 | 0 |
| 9 | [symbol] | 5 | 5 | 1 |
| 10 | [symbol] | 2 | 1 | 0 |
| 11 | [symbol] | 4 | 5 | 1 |
| 12 | [symbol] | 5 | 5 | 1 |
| 13 | [symbol] | 3 | 1 | 0 |
| 14 | [symbol] | 4 | 5 | 1 |
| 15 | [symbol] | 2 | 5 | 0 |
| 16 | [symbol] | 4 | 5 | 1 |
| 17 | [symbol] | 2 | 1 | 0 |
| 18 | [symbol] | 4 | 5 | 1 |
| 19 | [symbol] | 2 | 2 | 0 |
| 20 | [symbol] | 4 | 2 | 0 |
| 21 | [symbol] | 2 | 2 | 0 |
| 22 | [symbol] | 5 | 5 | 1 |
| 23 | [symbol] | 3 | 2 | 0 |
| 24 | [symbol] | 2 | 1 | 0 |
| 25 | [symbol] | 5 | 5 | 1 |
| 26 | [symbol] | 3 | 3 | 1 |
| 28 | [symbol] | 4 | 5 | 1 |
| 29 | [symbol] | 5 | 5 | 1 |
| 30 | [symbol] | 5 | 5 | 1 |
| 31 | [symbol] | 3 | 5 | 1 |
| 32 | [symbol] | 5 | 5 | 1 |

[symbol]

indicates data missing or illegible when filed

TABLE 3

| | Magnitude of | | Duration of rise | | Rise ratios | | Slope | | | |
|---|---|---|---|---|---|---|---|---|---|---|
| | increase (pg/mL) | | (hr) | | (fold increase) | | (pg/ml/hr) | | Abeta score | |
| Patient | Good | Poor | Good | Poor | Good | Poor | Good | Poor | Good | Poor |
| MA 230225 | | 18 | | | | 5 | | 0.61 | | |
| AA 800606 | | 6.8 | | 60 | | 35 | | 0.155 | | 101.8 |
| OL 510528 | 12 | | 31 | | 5 | | 0.316 | | 48 | |
| LS 550912 | 12.4 | | 24 | | 5 | | 0.517 | | 41.4 | |
| HT 490413 | | 4.6 | | 63 | | 5.6 | | 0.242 | | 73.2 |
| BK 321215 | | 8.6 | | 71 | | 9.6 | | 0.037 | | 89.2 |
| BE 470824 | 5.05 | | 30 | | 6.3 | | 0.168 | | 41.35 | |
| PU 330609 | 7.8 | | 10 | | 3.0 | | 0.6 | | 21.4 | |
| ES 370313 | | 14 | | 60 | | 8 | | 0.35 | | 82 |
| SP 350326 | 8.2 | | 12 | | 1.63 | | 0.656 | | 21.83 | |
| UE 450604 | 7.2 | | 25 | | 8.75 | | 0.266 | | 40.95 | |
| BJ 430420 | 11 | | 54.5 | | 6.5 | | 0.202 | | 72 | |
| KM 461001 | 4 | | 10 | | 2.3 | | 0.4 | | 16.3 | |
| JDK 371020 | | 14 | | | | 3.3 | | 0.93 | | |
| SW 480701 | 7.1 | | 1 | | 3.95 | | 0.546 | | 12.05 | |
| HW 450322 | 6.4 | | 12 | | 2.6 | | 0.256 | | 21 | |
| POL 290502 | | 9.1 | | | | 3.46 | | 0.758 | | |
| LP 470607 | | 12.3 | | 51 | | 8.7 | | 0.293 | | 72 |
| GJ 430818 | | 9.5 | | 84 | | 16.8 | | 0.174 | | 110.3 |
| ZD 330927 | | 6.8 | | | | 14.6 | | 0.358 | | |
| EG 500929 | | 14.4 | | 30 | | 7 | | 0.48 | | 51.4 |
| IJ 2 221008 | | 7.9 | | 63 | | 4.3 | | 0.208 | | 80.2 |
| NL 470320 | | 7.2 | | 56 | | 15.4 | | 0.141 | | 78.6 |
| AVG | 8.12 | 10.25 | 20.95 | 60.33 | 4.56 | 10.52 | 0.39 | 0.37 | 33.63 | 82.08 |

| | | | | | |
|---|---|---|---|---|---|
| t-test (P) | 0.081455564 | 0.000014 | 0.023243907 | 0.399662724 | 0.000009 |

TABLE 4

| t-test “Good” vs “Poor” 6 Mo Outcome | | P | |
|---|---|---|---|
| 1. Max slope of Ab42 rise | | 0.400 | |
| 2. Magnitude of Ab42 rise | | 0.0815 | |
| 3. Ab42 rise ratio | | 0.0232 | |
| 4. Duration of Ab42 rise | | 0.000014 | |
| 5. Overall Ab42 Score (sum of 2-4) | | 0.000009 | |
| | | 6 Mo Outcome | |
| | | Poor | Good |
| Ab42 score | <50 | 0 | 9 |
| | >50 | 9 | 1 |

Sensitivity for calling poor outcome: 9/9 (100%)
Specificity for calling good outcome: 9/10 (90%)

### Example 2

The following example describes a beta-amyloid (1-42) (i.e., Aβ42) ultra-sensitive digital immunoassay for plasma amyloid β42 using single molecule arrays.

Summary: Aβ42 has gained attention in the last 15 years as a biomarker correlating with Alzheimer's disease (AD) onset, mild cognitive impairment, vascular dementia, and other cognitive disorders. Substantial clinical data has validated the disease relevance of cerebral spinal fluid (CSF) levels of Aβ42, and there has been significant interest in measuring blood levels of the biomarker. However, the concentrations of Aβ42 in plasma are close to or below the limits of detection of most current immunoassay methods, making their precise determination challenging. A single molecule capable assay technology was utilized in this example to develop and validate an ultra-sensitive assay for measuring Aβ42 in plasma.

Reagents were developed for a paramagnetic bead-based ELISA. Aβ42 molecules in plasma were captured on antibody-coated paramagnetic capture beads and labeled with an enzyme conjugate. The beads were loaded into arrays of 50,000, 50-femtoliter reaction wells etched into bundles of optical fibers. Single capture beads trapped in each well were sealed in the presence of enzyme substrate and imaged using a fluorescence microscope fitted with a CCD camera. At low concentrations, the images were analyzed for the presence or absence of single immunocomplexes of labeled Aβ42, resulting in a digital signal. At high concentrations, the analog intensity of the beads was normalized to the digital signals, extending the dynamic range of the assay to over four logs. Analytical performance of the assay was evaluated, and plasma and CSF samples from Alzheimer's disease patients and normal control subjects were tested for Aβ42.

The assay described in this example has a detection limit (i.e., LOD) of 0.02 pg/mL and was linear to 250 pg/mL Aβ42 (R2>0.999). Recovery of Aβ42 spikes in plasma averaged 103% of expected. The assay exhibited no cross reactivity to shorter peptide variants Aβ38 and Aβ40, and 11-16% cross reactivity to the longer variant Aβ43. Intra-assay coefficients of variation (CV) were less than 10% down to 0.5 pg/mL. Total CVs (intra-assay and inter-assay) across six days of testing were 6.1%, 5.7%, and 10.0% for Aβ42 levels of 0.5, 1.0, and 5.0 pg/mL, respectively. The limit of quantification was estimated by testing ultra low panels of depleted plasma on multiple days. The total CVs were less than 20% for all ultra low panels tested (lowest panel 0.072 pg/mL), yielding an estimated limit of quantification (LOQ) of 0.042 pg/mL (0.189 fmol/L) using a criterion of 20%. Results using CSF samples from AD patients and age-matched normal control subjects showed excellent discrimination between the two populations, but results with plasma samples obtained from these same individuals showed no discrimination.

Further Description: Beta-amyloid peptides are 36-43 amino acid proteolytic products from the amyloid precursor protein that have gained considerable attention in the last 15 years as a biomarker correlating with Alzheimer's disease onset, mild cognitive impairment, vascular dementia, and other cognitive disorders Clinical validation has now been developed around disease relevance of cerebral spinal fluid levels of Aβ42, and there follows a significant interest in measuring blood levels of this biomarker. The interest originates in part from extensive pharmacologic efforts aimed at blocking production of beat-amyloid through inhibition of β-secretase and γ-secretase activating protein, and use of plasma measurements of amyloid β peptides as outcome indicators. Concentrations of Aβ42 in plasma are, however, over 100-fold lower than in cerebrospinal fluid. Recently reported was a blinded proficiency survey of several laboratories assaying plasma Aβ42 with different ELISA protocols. The survey showed high inter-lab variability in measured Aβ42 values and a broad range of intra-assay CVs; inter-assay CVs were not evaluated. A high-sensitivity ELISA for measurement of plasma Aβ42 has been reported, but potential therapeutic strategies aimed at lowering Aβ42 may put robust measurement of the peptide in blood beyond the reach of all current ELISA methods. To address this, a solid-phase extraction was recently developed and reverse-phase HPLC pre-treatment aimed at concentrating plasma levels of Aβ42 for improved immunoassay quantification. Enrichment of plasma Aβ42 by these procedures was found to lower the limit of detection to approximately 1 pg/mL, allowing quantification of the peptide in normal plasma at levels previously below the detection limit of even the high-sensitivity ELISA.

This example described the development and validation of a novel digital immunoassay using single molecule array technology that is capable of measuring Aβ42 in both normal and highly depleted plasma without biomarker enrichment or sample pretreatment procedures. The assay exhibits over 1000-fold greater sensitivity than validated commercially available ELISAs, and more than 50-fold greater sensitivity than what have been attainable using sample enrichment procedures described to date. The assay can be used for directly measuring and monitoring plasma Aβ42 in therapeutic trials aimed at altering and lowering levels of this peptide, down to sub-femtomolar levels.

The single molecule array technology employed two primary steps: a standard ELISA conducted with paramagnetic beads, followed by isolation of individual beads in arrays of femtoliter-sized reaction wells for digital imaging. Isolation of the individual beads in microwells permits the buildup of fluorescent product from the enzyme label such that signal from a single immunocomplex is readily detected using a CCD camera.

This approach permits counting of single molecules when Aβ42 concentrations are low enough that the ratio of bound labeled peptide per bead is much less than one. In this concentration realm, Poisson statistics predict that bead-containing microwells in the array will contain either a single labeled Aβ42 molecule or no labeled Aβ42 molecules, resulting in a binary signal. Due to the amplified sensitivity for detecting label molecules afforded by confining fluorescent product buildup to the microwells, concentrations of label (detector anti-Aβ42 antibody and enzyme label) can be reduced relative to standard ELISAs. Lowered concentrations of labeling reagents reduces their interaction with capture beads, resulting in reduced nonspecific binding enabling high signal to background ratios, even at extremely low concentrations of biomarker. For higher biomarker concentrations where all beads contain one or more labeled immunocomplexes, digital signals from the Poisson realm are used to calibrate analog intensity measurements, extending the dynamic range to over four logs.

Three reagents were developed for this Aβ42 immunoassay: capture beads, biotinylated detector, and a conjugate of streptavidin:beta-galactosidase. The capture bead reagent comprised of a commercially available monoclonal anti Aβ42 antibody (Covance) directed to a N-terminus epitope (amino acids 3-8). The antibody was covalently attached by standard coupling chemistry to 2.7 μm carboxy paramagnetic microbeads (Varian). Because individual beads were captured in array wells 4.5 μm wide×3.25 μm deep, it was important that the capture beads remain monomeric. The antibody-coated beads were diluted to a working concentration of 5×106 beads/mL in Tris buffer with a surfactant and BSA. The biotinylated detector reagent was comprised of a commercially available monoclonal anti Aβ42 antibody (Invitrogen) directed to the C-terminus of the peptide. The antibody was biotinylated using standard methods (Solulink), and the biotinylation level was confirmed spectrophotometrically per manufacturer's instructions. The monomeric state of the detector antibody before and after biotinylation was confirmed by size exclusion HPLC. The biotinylated detector antibody was diluted to a concentration of 0.1 μg/ml for assay in a PBS diluent containing a surfactant and newborn calf serum, NCS (PBS/NCS). The enzyme conjugate-streptavidin:β-galactosidase (SBG)-was prepared by covalent conjugation of purified streptavidin (Thermo Scientific) and β-galactosidase (Sigma) using standard coupling chemistry (1-ethyl-3-(3-dimethylaminopropyl) carbodiimide hydrochloride, Thermo Scientific). Aliquots of a concentrated stock solution of SBG were prepared in PBS with 50% glycerol and transferred to −20° C. for storage. Prior to assay, an aliquot was thawed and diluted to 25 pM in PBS/NCS with MgCl₂. Purified Aβ42 antigen for calibrators and peptide variants Aβ38, Aβ40, and Aβ43 for specificity testing were from Merck.

Assay calibrators and controls were prepared by dilution of Aβ42 stock in PBS diluent containing a surfactant and BSA (PBS/BSA). In some cases, a stock solution prepared by dilution to 3.5 ng/ml in PBS/Tween-20. For dynamic range/linearity characterization, a series of calibrators were prepared by serial 3-fold dilution to give a calibration range of 0-250 pg/mL. To evaluate assay day-to-day reproducibility at low concentrations, three low controls were prepared at 0.5, 1.0, and 5.0 pg/mL in PBS/BSA. To estimate the limit of quantification (LOQ), three ultra-low plasma controls were prepared using a pool of immunodepleted normal human EDTA plasma. Immunodepletion was accomplished by incubation overnight at 2-8° C. with shaking of 5 mL of plasma with 1×109 Aβ42 capture beads prepared as described above. The immunodepleted plasma was used as an ultra low plasma control, along with two additional ultra-low controls of immunodepleted plasma spiked with trace amounts of Aβ42.

50,000 well optical fiber microarrays were prepared as previously described. In brief, optical fiber bundles (Schott North America) were cut into 5 cm lengths and sequentially polished with 30, 9, and 1 um-sized diamond lapping films. One end of each bundle was etched in a 0.025 N HCl solution for 2 minutes and then submerged into water. The differential etch rate of the core and cladding glass of the fiber bundles caused an array of 4.5 um diameter wells to be formed in the core fibers.

Bead-plasma incubations and labeling of immunocomplexes in conical 96 well plates (Axygen) were conducted using a robotic liquid handling system (Tecan EVO 150). Conical wells are used to facilitate magnetic attraction of the beads to the sides of the wells for efficient removal of reaction mixtures and bead washing. For magnetic attraction, a microplate bar magnet (Invitrogen) was used. Incubation periods were conducted with shaking on a microplate shaker (VWR) to keep beads suspended in the wells. The assays were initiated by mixing 100 uL of sample with 500,000 capture beads, and the mixtures were incubated with shaking for two hours. Plasma samples were pre-diluted 1:4 prior to assay with PBS/BSA as a precaution for sample quality and interference effects. Following incubation, the beads were washed 3 times with a wash buffer of 5-fold concentrated PBS with a surfactant (5×PBS). 100 uL of biotinylated detector antibody was then added and incubated with the beads for 45 minutes. After a second sequence of three washes with 5×PBS, 100 μL of streptavidin-β-galactosidase was incubated with the beads for 30 minutes to form the final enzyme-labeled immunocomplex. The beads were then washed six times per above, and concentrated to 2×10⁷ beads/ml with the addition of a reduced volume (25 μL) of array loading buffer comprised of PBS with a surfactant. Beads were then loaded onto the arrays. 10 μL of the concentrated bead solution (2×10⁶ beads) were pipetted onto the arrays and the arrays were centrifuged at 1,300 g for 10 minutes. Excess beads were removed by a PBS rinse and swabbing with deionized water. With this technique, array filling by the beads was generally 50-60%, which was adequate for minimizing contributions to imprecision from Poisson noise. Wells containing labeled Aβ42-labeled beads were detected utilizing β-galactosidase catalyzed hydrolysis of resorufin β-D-galactopyranoside (RGP, Invitrogen) into fluorescent product (resorufin, excitation 558 nm, emission 577 nm). To introduce RGP substrate to the array wells, droplets of substrate were placed on a silicone gasket and introduced into the array wells with a mechanical platform. This step resulted in an array of sealed femtoliter wells in which enzyme-containing beads developed a concentrated fluorescence signal.

Imaging was accomplished via a custom-built fluorescence imaging system containing a light source, objectives, filter cubes and a CCD camera. For each sample, five fluorescent images of one second each were acquired (to identify wells containing an enzyme) and one white light image was acquired (to identify wells containing a bead). Background fluorescence and any contaminating artifacts were discriminated from true ‘positive’ wells by analyzing for signal growth over the multiple images. These images were analyzed to determine the average number of enzymes per bead (AEB) across the concentration range. In some cases, at <50% active beads, the system was determined to be in the digital realm, so AEB was be determined from the fraction of beads that contain at least one enzyme and the Poisson distribution; and at >50% active beads, the average fluorescence intensity of the beads was normalized to the average fluorescence intensity of beads containing a single enzyme to yield AEB. In other cases, at <70% active beads relative to total beads, AEB was determined as a count of active beads corrected for a low statistical probability of multiple enzymes per bead; and at >70% active beads, the probability of multiple enzymes/bead increases such that all wells contain multiple enzymes and all are growing in signal and in this realm, the signal is was no longer digital, and average fluorescence intensities of the wells were converted to AEB based on the average intensities of wells containing single enzymes as determined at lower Aβ42 concentrations. The AEB unit worked continuously across the digital and analog realms.

For description of various details associate with this assay, see, for example, U.S. patent application Ser. No. 12/731,130, filed Mar. 24, 2010, published as US-2011-0212848 on Sep. 1, 2011, entitled “ULTRA-SENSITIVE DETECTION OF MOLECULES OR PARTICLES USING BEADS OR OTHER CAPTURE OBJECTS,” by Duffy et al.; International Patent Application No. PCT/US2011/026645, filed Mar. 1, 2011, published as WO 2011/109364 on Sep. 9, 2011, entitled “ULTRA-SENSITIVE DETECTION OF MOLECULES OR PARTICLES USING BEADS OR OTHER CAPTURE OBJECTS,” by Duffy et al.; International Patent Application No. PCT/US2011/026657, filed Mar. 1, 2011, published as WO 2011/109372 on Sep. 9, 2011, entitled “ULTRA-SENSITIVE DETECTION OF MOLECULES USING DUAL DETECTION METHODS,” by Duffy et al.; U.S. patent application Ser. No. 12/731,135, filed Mar. 24, 2010, published as US-2011-0212462 on Sep. 1, 2011, entitled “ULTRA-SENSITIVE DETECTION OF MOLECULES USING DUAL DETECTION METHODS,” by Duffy et al.; International Patent Application No. PCT/US2011/026665, filed Mar. 1, 2011, published as WO 2011/109379 on Sep. 9, 2011, entitled “METHODS AND SYSTEMS FOR EXTENDING DYNAMIC RANGE IN ASSAYS FOR THE DETECTION OF MOLECULES OR PARTICLES,” by Rissin et al.; U.S. patent application Ser. No. 12/731,136, filed Mar. 24, 2010, published as US-2011-0212537 on Sep. 1, 2011, entitled “METHODS AND SYSTEMS FOR EXTENDING DYNAMIC RANGE IN ASSAYS FOR THE DETECTION OF MOLECULES OR PARTICLES,” by Duffy et al.; U.S. patent application Ser. No. 13/035,472, filed Feb. 25, 2011, entitled “SYSTEMS, DEVICES, AND METHODS FOR ULTRA-SENSITIVE DETECTION OF MOLECULES OR PARTICLES,” by Fournier et al.; U.S. patent application Ser. No. 13/037,987, filed Mar. 1, 2011, published as US-2011-0245097 on Oct. 6, 2011, entitled “METHODS AND SYSTEMS FOR EXTENDING DYNAMIC RANGE IN ASSAYS FOR THE DETECTION OF MOLECULES OR PARTICLES,” by Rissin et al.; each herein incorporated by reference.

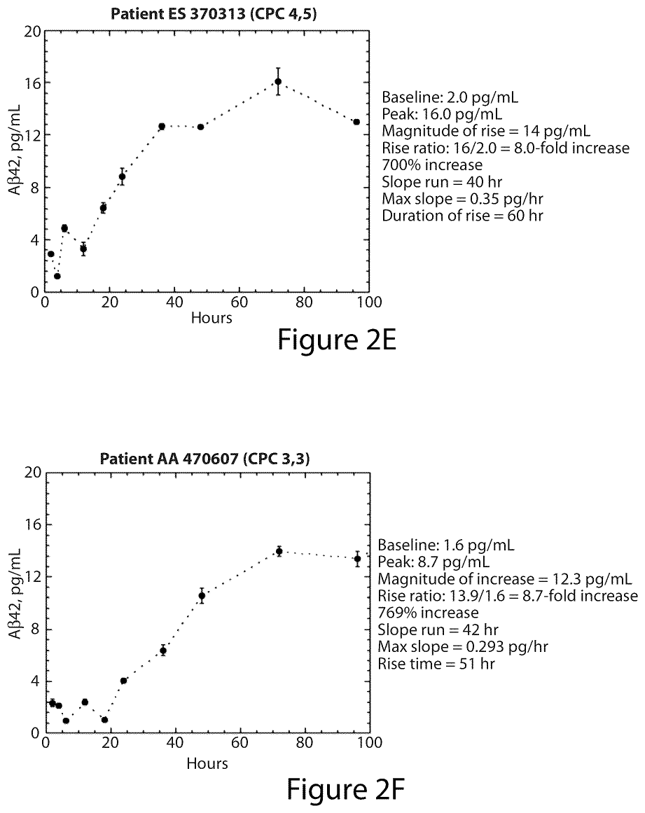

FIG. 4 shows a representative calibration dose-response of the Aβ42 assay up to 250 pg/mL. The assay demonstrated a highly linear response, with a linear regression R² of 0.9993. The inset highlights the low background at very low Aβ42 concentrations. In a study of calibration curves obtained on six different days, the mean signal to noise ratio at 1.0 pg/mL was 15.7 (SD 2.2). Limits of detection (3SD method) across the six days ranged from 0.014 to 0.032 pg/mL, with an average of 0.020 pg/mL (0.09 fmol/L), representing greater than four logs of assay range. In particular, FIG. 4 shows a representative dose-response of the digital Aβ42 immunoassay. Y-axis refers to the average number of label enzymes per individual microbead captured in the microwells of the optical fiber array. The inset highlights the low background obtained with single-molecule counting and digital quantification of extremely low analyte levels. Analysis of six separate calibration curves gave a mean signal to noise ratio at 1.0 pg/mL of 15.7 (SD 2.2).

Linearity in the extreme low end of the assay range was further evaluated by a series of four-fold dilutions (to 256-fold) of depleted plasma that had been spiked with 4 pg/mL Aβ42. The average recovery across the dilutions was within 0.02 pg/mL of the expected values. At the final 256-fold dilution, the assay measured an Aβ42 value of 0.118 pg/mL as compared with an expected value 0.099 pg/mL.

Recovery was evaluated by spiking 25 pg/mL Aβ42 into four normal plasma samples as well as the depleted plasma pool. Recoveries ranged from 91.5-113.4%, with an average of 103.6%.

Specificity of the assay for Aβ42 was evaluated by assaying 0.5, 1.0, 5.0, 10 and 50 pg/mL of the peptide variants Aβ38, Aβ40, and Aβ43 in PBS/BSA. No detectable cross reactivity was noted for the shorter Aβ38 and Aβ40 peptides, while the longer Aβ43 variant exhibited a cross reactivity of 11-16%.

Limits of detection (LOD) were estimated as three standard deviations above the zero calibrator across calibration curves on six separate days (n=3 replicates per curve). LODs ranged from 0.014 to 0.032 pg/mL, with an average of 0.020 pg/mL (0.09 fmol/L).

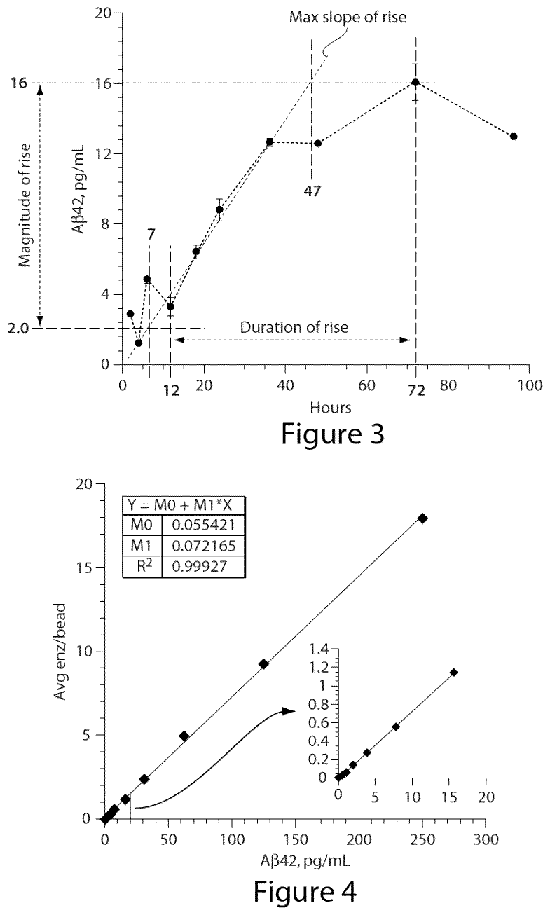

Limit of quantification (LOQ) was estimated with guidance from NCCLS EP17-A [20]. Total CVs from low panel members assayed on multiple days (Table 5) were plotted vs. their respective measured value (FIG. 5). The analyte concentration at which the assay's total measurement variation crossed 20% CV was taken to represent an estimated LOQ. Because none of the total CVs exceeded 20%, the 20% threshold was extrapolated from the equation of a non-linear power fit of the data (R²=0.703, KaleidaGraph). A LOQ of 0.032 pg/mL (0.144) was estimated at the 20% threshold.

TABLE 5
Study 1: Within-run Imprecision

| | pg/mL Aβ42 | | | | | | | | |
|---|---|---|---|---|---|---|---|---|---|
| Panel | result 1 | result 2 | result 3 | result 4 | result 5 | result 6 | AVG | SD | CV |
| L1 | 0.41 | 0.4 | 0.35 | 0.36 | 0.37 | 0.37 | 0.38 | 0.026 | 6.8% |
| L2 | 0.83 | 0.74 | 0.69 | 0.75 | 0.69 | 0.70 | 0.73 | 0.056 | 7.7% |
| L3 | 3.82 | 4.04 | 3.81 | 3.45 | 3.38 | 4.18 | 3.78 | 0.316 | 8.4% |

Study 2: Total Imprecison

| | pg/mL Aβ42 | | | | | | | | |
|---|---|---|---|---|---|---|---|---|---|
| Panel | day 1 | day 2 | day 3 | day 4 | day 5 | day 6 | AVG | SDₜₒₜ | CVₜₒₜ |
| L1 | 0.46 | 0.51 | 0.52 | 0.45 | 0.45 | 0.48 | 0.48 | 0.029 | 6.1% |
| L2 | 0.94 | 0.97 | 0.98 | 0.92 | 1.04 | 0.89 | 0.95 | 0.054 | 5.7% |
| L3 | 3.99 | 5.22 | 5.25 | 4.54 | 5.00 | 4.70 | 4.78 | 0.479 | 10.0% |

Study 3: Total Imprecision-Ultra Low Plasma

| | pg/mL Aβ42 | | | | | | | | |
|---|---|---|---|---|---|---|---|---|---|
| Panel | day 1 | day 2 | day 3 | day 4 | day 5 | day 6 | AVG | SDₜₒₜ | CVₜₒₜ |
| Depleted | 0.069 | 0.056 | 0.069 | 0.079 | 0.087 | ND | 0.072 | 0.012 | 16.3% |
| L2 | 0.151 | 0.128 | 0.123 | 0.110 | 0.118 | ND | 0.126 | 0.015 | 12.3% |
| L3 | 0.409 | 0.333 | 0.459 | 0.471 | 0.434 | ND | 0.421 | 0.055 | 13.0% |

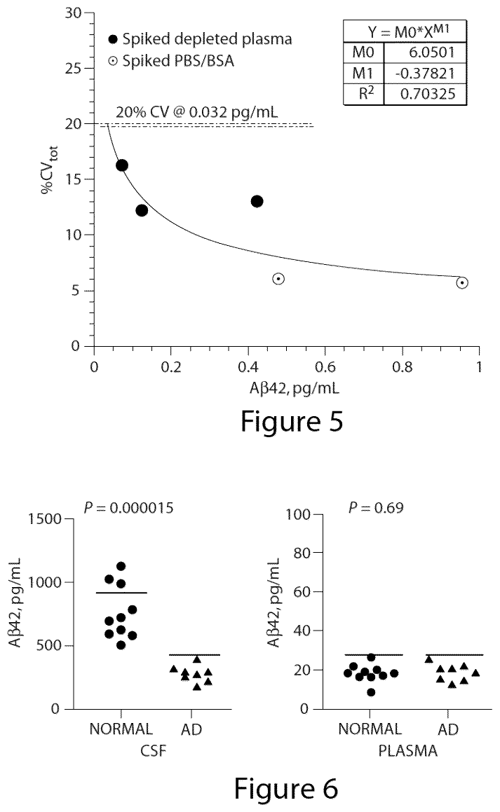

To further validate the assay, a pilot study of matched EDTA plasma and CSF samples from eight patients diagnosed with AD and nine normal individuals were assayed with the Aβ42 test (FIG. 6). Normal and disease specimens were matched with respect to age, gender, and collection dates. Patients were diagnosed with AD according to clinical criteria. The clinical work-up also included imaging and MMSE cognitive scoring criteria. Normal age matched individuals also received MMSE testing to ensure high cognitive functioning. All samples were collected under IRB approval and obtained from PrecisionMed SAMPLE (Serial Alzheimer's Disease and MCI Prospective Longitudinal Evaluation) repository. The results were consistent with previous data reported for both sample types. With CSF samples, excellent discrimination (P=0.000015) was observed between AD subjects and normal subjects. Results for the AD CSF specimens ranged from 192.1-333.6 pg/mL, and normal CSF specimens ranged from 509.9 to 1127.8 pg/ml. On the other hand, results from the plasma specimens lacked discrimination between AD and normal subjects (P=0.69). Aβ42 levels in AD plasma ranged from 12.6 to 25.8 pg/mL, while results for the normal plasma specimens ranged from 8.8 to 22.1 pg/mL, in agreement with reported ELISA values following solid phase extraction and HPLC enrichment.

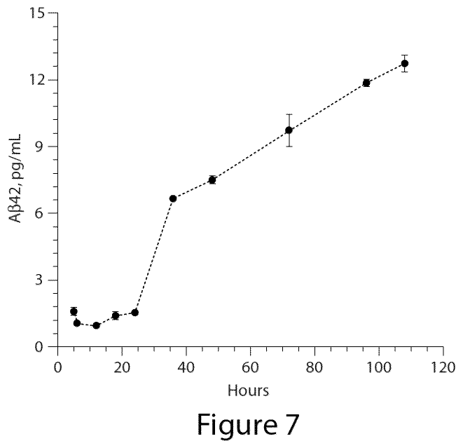

To test the assay performance in another clinical context, serial serum samples from a 41-year old man who was resuscitated after cardiac arrest with restoration of spontaneous circulation were analyzed. Samples were measured in triplicate in the Aβ42 assay. FIG. 7 depicts the profile of the serum Aβ42 change and shows a dramatic increase after the first 24 hours. Data from this study are the first to directly link hypoxic stress to AB elevation in humans. The Aβ42 concentrations measured in this study, particularly during the first 24 hours, were below the measurement capability of commercially available immunoassays, illustrating the potential utility of SiMoA technology as a new tool for precise measurement of low-abundance Aβ42 and other protein biomarkers.

Discussion. The data presented here indicate that the Aβ42 assay represents a capability for extremely sensitive and reproducible Aβ42 measurement in serum and plasma. The assay allows highly sensitive (limit of quantification 0.032 pg/mL) and precise quantification of Aβ42 in biological fluids.

Current immunoassays for measurement of Aβ42 in plasma are based on the ELISA or the Luminex techniques. In addition to lower analytical sensitivity, these immunoassays are hampered by low recovery of spiked Aβ42 into plasma or serum and as a result the measured concentration of Aβ42 increases when the plasma sample is diluted. These phenomena are likely due to matrix effects, probably by interaction and binding of Aβ42 to plasma proteins. This means that unrelated plasma proteins contribute to the concentration interpreted to come from Aβ42 in this type of assay. The immunoassay presented here showed highly linear dilution characteristics, together with a recovery of around 100%, suggesting that it gives a very accurate measure of the Aβ42 concentration. This is likely because the interference from plasma proteins is lost when samples are pre-diluted 1:4 prior to measurement. Because the assay has such high sensitivity, pre-diluting the samples does not compromise the capability of the method to precisely measure low abundance Aβ42.

Conflicting results have been reported regarding the association of plasma levels of Aβ42 with AD. For example, one report reported a statistically significant decrease in plasma Aβ42 in a cohort of 149 AD patients relative to 89 age-matched controls (mean 38 and 52 pg/mL respectively, p<0.01), although there was large overlap between the two populations. In another case, no discernable difference in plasma Aβ42 between sporadic AD patients (n=78) and age-matched controls (n=61) was reported. Use of different ELISAs and analytical noise could have contributed to lack of agreement. It is also clear that most previously available assays measure Aβ42 close to the limit of quantification, which may contribute to variation and inconsistent results between studies. If there is a decline in plasma Aβ42 with AD, it appears to be modest, requiring statistically powered studies to discern. Because depression of measurable Aβ42 in CSF relative to controls is a well established hallmark of AD (also seen here), the apparent disconnect between CSF and plasma results can led to the suggestion that the normal equilibrium between CSF and plasma may be disrupted with the initiation of amyloid deposition in the brain. The study reported here was intended to confirm expected performance of the Aβ42 assay with plasma and CSF clinical samples. The data were consistent with expected results from both sample types.

The Aβ42 assay described here was able to precisely monitor serum Aβ42 changes in a survivor of oxygen deprivation. Cerebral ischemia is believed to play a role in AD pathogenesis. The finding of a significant increase in serum Aβ42 following oxygen deprivation is the first direct demonstration of a link between hypoxic stress and AB elevation in humans. Nevertheless, the kinetic profile depicted in FIG. 7 could reflect proteolysis of amyloid precursor protein by β-secretase (BACE1), in which BACE1 is upregulated by the transcription factor hypoxia-inducible factor-1. Animal models have helped elucidate this pathway, and detecting hypoxia-driven amyloid cascade in humans could have significance for further understanding this process and its role in AD pathogenesis.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used.

Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements.

This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively. as set forth in the United States Patent Office Manual of Patent Examining Procedures, Section 2111.03.

What is claimed:

1-99. (canceled)

100. A method of improving computer implemented analysis of patient data for determining a prognosis for a neurological condition, comprising a computer implemented method for determining a series of beta-amyloid profile parameters predictive of a prognosis for a patient's recovery from the neurological condition, from a data set obtained from performing an assay on a plurality of blood samples from the patient; the assay comprising:
performing an assay on each of a plurality of blood samples obtained from the patient suspected of having the neurological condition and/or plasma and/or serum derived from each of the blood samples to determine a measure of a beta-amyloid peptide concentration in the sample, wherein a collection of beta-amyloid peptide molecule concentrations as a function of collection time for the plurality of samples comprises the data set;
wherein the assay comprises spatially segregating and detecting single beta-amyloid peptide molecules,
wherein the limit of detection of beta-amyloid peptide of the assay is less than about 0.2 pg/mL;
wherein the computer implemented method for determining a series of beta-amyloid profile parameters comprises:
generating from the data set:
Profile Parameter (1): a magnitude of rise in beta-amyloid peptide concentration;
Profile Parameter (2): a duration of rise of beta-amyloid peptide concentration; and
Profile Parameter (3): a ratio of the peak beta-amyloid peptide concentration to a minimum beta-amyloid peptide concentration; and
wherein the method of improving computer implemented analysis of patient data for determining a prognosis for the neurological condition comprises:
(a) computing an overall beta-amyloid score based on values of Profile Parameters (1)-(3) by combining all three values of the Profile parameters (1)-(3), and
(b) implementing the beta-amyloid score for determining a prognosis for the patient for the neurological condition.

101. The method of claim 100, wherein the single beta-amyloid peptide molecules are spatially segregated into a plurality of locations, wherein a statistically significant fraction of the locations contain a single molecule of the beta-amyloid peptide and a statistically significant fraction of the locations do not contain any molecules of the beta-amyloid peptide.

102. The method of claim 101, wherein the assay further comprises addressing at least a portion of the plurality of locations subjected to the spatially segregating step and determining the number of said locations containing a beta-amyloid peptide molecule.

103. The method of claim 102, wherein the plurality of locations comprises a plurality of reaction vessels.

104. The method of claim 103, wherein the average volume of the plurality of reaction vessels is between about 10 attoliters and about 100 picoliters.

105. The method of claim 103, wherein the average volume of the plurality of reaction vessels is between about 1 femtoliter and about 1 picoliter.

106. The method of claim 100, wherein the single beta-amyloid peptide molecules are immobilized with respect to capture objects.

107. The method of claim 106, wherein the capture objects comprises beads.

108. The method of claim 100, wherein the patient suspected of having a neurological condition is recovering from a brain injury.

109. The method of claim 108, wherein the plurality of samples are collected within 96 hours after the patient has experienced the brain injury or suspected brain injury.

110. The method of claim 108, wherein the brain injury results from a hypoxic event.

111. The method of claim 100, wherein the beta-amyloid peptide is beta-amyloid (1-42).

112. The method of claim 100, wherein the assay is performed on the blood samples obtained from the patient.

113. The method of claim 100, wherein the assay is performed on plasma derived from the blood samples obtained from the patient.

114. The method of claim 100, wherein the assay is performed on serum derived from the blood samples obtained from the patient.

115. The method of claim 100, wherein the patient's prognosis is correlated with a Cerebral Performance Category (CPC) score, wherein the CPC score includes classifications of good or poor, wherein a good CPC score indicates a prognosis of the patient being conscious and alert with normal function or only slight disability or conscious and alert with moderate disability after a selected period of time, and wherein a poor CPC score indicates a prognosis of the patient being conscious with the patient having a severe disability, being comatose or in a persistent vegetative state, or being brain dead or suffering death from other causes after a selected period of time.

116. The method of claim 100, wherein Profile Parameter (1) is generated from a plot of the measure of the beta-amyloid peptide concentration in the plurality of samples as function of time.

117. The method of claim 100, wherein Profile Parameter (1) is measured in pg/mL, Profile Parameter (2) is measured in hours, and wherein the prognosis is good for the recovery from the brain injury if the sum is less than about 50.

118. The method of claim 100, wherein Profile Parameter (1) is measured in pg/mL, Profile Parameter (2) is measured in hours, and wherein the prognosis is poor for the recovery from the brain injury if the sum is greater than about 50.
